(19) United States
(12) Patent Application Publication
(10) Pub. No.: US 2021/0285951 A1
(43) Pub. Date: Sep. 16, 2021

(54) COMPOSITIONS AND METHODS RELATING TO INHIBITORS OF PRO-INFLAMMATORY CYTOKINES AND CHEMOKINES FOR TREATMENT OF CANCER

(71) Applicant: Wayne State University, Detroit, MI (US)

(72) Inventor: Isaac J. Powell, Detroit, MI (US)

(21) Appl. No.: 17/200,204

(22) Filed: Mar. 12, 2021

Related U.S. Application Data
(60) Provisional application No. 62/988,656, filed on Mar. 12, 2020.

(51) Int. Cl. G01N 33/574; A61K 45/06; G01N 33/68

(52) U.S. Cl. CPC G01N 33/57434; G01N 2800/56; G01N 33/68; A61K 45/06

(57) ABSTRACT

Methods of treatment of cancer, including prostate cancer, in a subject in need thereof are provided according to aspects of the present disclosure which include administering an effective amount of an inhibitor of a pro-inflammatory factor to the subject, wherein the pro-inflammatory factor is dysregulated in the subject, and wherein the inhibitor is an inhibitor of a pro-inflammatory factor selected from the group consisting of: interleukin-1β, interleukin-6, interleukin-8, interleukin-10, stromal cell-derived factor-1α, matrix metallopeptidase 9, epithelial-derived neutrophil-activating peptide 78, receptor for advanced glycation end products, TNF-α, HER2, CXCR4, and leptin. Assays of these factors, or subsets thereof, provide a pro-inflammatory cytokine score useful in determining a characteristic of the cancer, particularly aggressiveness.

### RELATED APPLICATIONS

The present application claims priority to U.S. Provisional Patent Application 62/988,656, filed Mar. 12, 2020, which is hereby incorporated by reference in its entirety.

### FIELD OF THE INVENTION

This disclosure relates generally to compositions and methods for inhibiting pro-inflammatory factors, including cytokines and chemokines, receptors thereof, and stimulators thereof, for treatment of cancer. According to specific aspects, this disclosure relates to compositions and methods for inhibiting pro-inflammatory factors including cytokines and chemokines, receptors thereof, and stimulators thereof, for treatment of prostate cancer.

### BACKGROUND OF THE INVENTION

Prostate cancer is the most common malignancy in men and the second leading cause of death from cancer in the United States second only to lung cancer, killing about 27,540 men each year. About 1 man in 38 will die of prostate cancer. Prostate cancer represents 13.3% of all new cancer cases in the U.S., with about 220,800 new cases of prostate cancer reported each year. About 1 in 7 men will be diagnosed with prostate cancer during their lifetime. The average age at the time of diagnosis is about 66 years old.

The relative 5-year survival rate for prostate cancer is nearly 100%; however, the survival rate for those diagnosed as stage IV with distant metastases is only 28%. Despite more aggressive screening across all demographics and gradual declines in mortality related to prostate cancer in the United States, disparities among populations persist. A substantial proportion of men of African descent have a higher overall incidence, earlier age of onset, increased proportion of clinically advanced disease, and increased bone metastases and mortality from prostate cancer (PCa) compared to men of European descent.

Aggressive prostate cancer leads to a higher metastasis rate and requires early detection and treatment. Since the discovery of prostate-specific antigen (PSA), assays that detect this serum biomarker (together with digital rectal exams) have been used for the screening of prostate cancer. Current initial prostate cancer diagnosis is typically by a prostate biopsy after an abnormal digital rectal exam (DRE) or detection of elevated PSA. PSA testing combined with DRE helps identify prostate cancers at their earliest stages, but studies have disagreed whether these tests reduce the risk of dying of prostate cancer. For that reason, there is debate surrounding prostate cancer screening. If an abnormality is detected on a DRE or PSA test, a physician may recommend tests to determine whether you have prostate cancer, such as ultrasound or biopsy. Although PSA testing has resulted in early detection and intervention, the major limitation of PSA is the low specificity and high prevalence of detecting benign prostatic hyperplasia, especially in older men. Early detection based on PSA testing also fails to distinguish aggressive prostate cancer from non-aggressive prostate cancer. Among men treated for prostate cancer, increasing prostate-specific antigen PSA is known as biochemical failure or biochemical recurrence (BCR). The impact of BCR on subsequent mortality is uncertain, however, especially given competing causes of death. Indeed, with the illustration of the limitations of the current PSA-based screening method, a published study randomly assigned 76,693 men at 10 U.S. study centers to receive either annual PSA screening (38,343 subjects) or usual care as the control (38,350 subjects); this study reported no statistical differences in prostate cancer specific mortality between the groups after 7-10 years of follow-up.

The use of PSA screening has resulted in a stage shift to early prostate cancer and in many cases, low risk prostate cancer. There is considerable controversy as to whether “low risk” prostate should be treated. It is increasingly clear that some low risk prostate cancer patients do not progress to aggressive disease and do not need treatment whereas others progress and require treatment; however, it remains difficult to predict which patients will progress from those who will not using histological and clinical characteristics. Currently, there is an increasing use of active surveillance to prevent over-treatment. A study conducted in the state of Michigan by the MUSIC (Michigan Urological Surgery Improvement Collaborative), found that 50% of men with low risk prostate cancer are placed on active surveillance (Womble et al., Eur. Urol., 2015, 67(1):44-50). Yet there are at least three reports that support caution in including young men of African descent in surveillance, particularly considering that men of African descent are 3-fold more likely than men of European descent to have disease progression. Iremashvili et al. (J. Urol., 2012, 187(5):1594-9) reported that 26% of their patients showed progression at a median of 2.9 year follow up on a mean of 2.3 surveillance biopsies. The progression risk was significantly increased in patients of African descent (adjusted HR 3.87-4.12), and in men with a smaller prostate and higher prostate specific antigen density. Iremashvili et al. concluded that men of African descent with “low risk” prostate cancer should be advised that the risk of progression on active surveillance many be higher than that in the available literature. One study reported that men of African descent with very low-risk prostate cancer had more adverse pathologic features at radical prostatectomy and poorer oncologic outcomes (Sundi et al., J. Clin. Oncol., 2013, 20; 31(24):2991-7). Men of African descent were more likely to experience disease upgrading at prostatectomy (27.3% vs 14.4%; P<0.001, positive surgical margins (9.8% vs 5.9%; P=0.02, and higher Cancer of the Prostate Risk Assessment Post-Surgical scoring system (CAPRA-S) scores. On multivariable analysis, the African American race was an independent predictor of adverse pathologic features (odds ratio, [OR] 3.23; P=0.03 and pathologic upgrading (OR, 2.26; P=0.01). Another study reported that African descent was associated with discontinuation of active surveillance for treatment. Men of African descent were associated with treatment (hazard ratio (HR) 2.93, P=0.01) as compared with men of European descent (Abern et al., Prostate Cancer Prostatic Dis., 2013, 16(1):85-90). When the analysis was adjusted for socio-economic and clinical parameters at the time of prostate cancer diagnosis, men of African descent remained the sole predictor of treatment (HR 3.08, P=0.01). Among men undergoing treatment, the trigger was less often patient driven in men of African descent compared to men of European descent P=0.05 (Abern et al., 2013). As stated earlier, data was reported that prostate cancer grows faster among men of African descent compared to men of European descent. Researchers at Johns Hopkins are reporting long term follow up of racial disparities in oncologic outcomes after radical prostatectomy. In findings using biochemical recurrence (BCR) as an endpoint, men of African descent with very low, low or intermediate risk prostate cancer who undergo radical prostatectomy are more likely to have adverse pathologic findings and BCR compared to men of European descent. Even more important, the data show that BCR-free survival for low risk men of African descent is similar to intermediate risk men of European descent (Faisal A., BJU Int., 2014, 114(6b):E120-E129)

Similarly, other current means of prostate cancer risk assessment are also too imprecise to be useful due to determine whether “low risk” prostate patients will progress from those who will not, and thus who should be treated. For example, to determine if a prostate cancer is aggressive (grade), a common scale called the Gleason Score is commonly used. A pathologist microscopically examines a biopsy specimen for certain “Gleason” patterns. These Gleason patterns are associated with the following features: Pattern 1—The cancerous prostate closely resembles normal prostate tissue. The glands are small, well-formed, and closely packed. This corresponds to a well differentiated carcinoma. Pattern 2—The tissue still has well-formed glands, but they are larger and have more tissue between them, implying that the stroma has increased. This also corresponds to a moderately differentiated carcinoma. Pattern 3—The tissue still has recognizable glands, but the cells are darker. At high magnification some of these cells have left the glands and are beginning to invade the surrounding tissue or having an infiltrative pattern. This corresponds to a moderately differentiated carcinoma. Pattern 4—The tissue has few recognizable glands. Many cells are invading the surrounding tissue in neoplastic clumps. This corresponds to a poorly differentiated carcinoma. Pattern 5—The tissue does not have any or only a few recognizable glands. There are often just sheets of cells throughout the surrounding tissue. This corresponds to an anaplastic carcinoma.

A pathologist then assigns a grade to the observed patterns of the tumor specimen. A primary grade is assigned to the dominant pattern of the tumor (has to be greater than 50% of the total pattern seen). A secondary grade is assigned to the next-most frequent pattern (has to be less than 50%, but at least 5%, of the pattern of the total cancer observed). The pathologist then sums the pattern-number of the primary and secondary grades to obtain the final Gleason score. If only two patterns are seen, the first number of the score is that of the tumor's primary grade while the second number is that of the secondary grade, as described in the previous section. If three patterns are seen, the first number of the score would be the primary grade and the second number the pattern with the highest grade. However, the risk assessment based on this clinical criterion is too imprecise to be useful due to biopsy sampling error and interobserver grading differences. It is also unable to be used as a non-invasive screening test for early detection of aggressive prostate cancer.

Due to the above mentioned deficiencies in currently available screening methods, aggressive prostate cancer is under detected and under treated while nonaggressive prostate cancer is over detected and over treated. Additionally, the incidence of prostate cancer is 60% greater and the mortality rate is 2 to 3 times higher when comparing men of African descent with men of European descent.

There is a continuing need for identification of factors and biomarkers that are differentially present in non-aggressive prostate cancer and aggressive prostate cancer, as well as differentially present in men of African descent and men of European descent, along with methods of treatment of such cancers.

### SUMMARY OF THE INVENTION

It is understood that both the following summary and the detailed description are exemplary and explanatory and are intended to provide further explanation of the disclosure as claimed. Neither the summary nor the description that follows is intended to define or limit the scope of the disclosure to the particular features mentioned in the summary or description.

Methods of treatment of a subject having, or suspected of having, cancer are provided according to aspects of the present disclosure which include: inhibiting two or more of: CXCR4, IL-8, IL-6, HER2, TNFα, IL-1β, SDF-1a, MMP-9, ENA-78, leptin, RAGE, and IL-10 in the subject, with the proviso that, when CXCR4 and TNFα are inhibited, at least one additional member of the group: IL-8, IL-6, HER2, IL-1β, SDF-1a, MMP-9, ENA-78, leptin, RAGE, and IL-10 is also inhibited; with the proviso that, when CXCR4 and SDF-1a are inhibited, at least one additional member of the group: IL-8, IL-6, HER2, IL-1β, TNFα, MMP-9, ENA-78, leptin, RAGE, and IL-10 is also inhibited; with the proviso that, when IL-8, IL-6, and TNFα are inhibited, at least one additional member of the group: CXCR4, HER2, SDF-1a, MMP-9, ENA-78, leptin, RAGE, and IL-10 is also inhibited; and with the proviso that when IL-8, IL-6, TNFα, and IL-1β are inhibited, at least one additional member of the group: CXCR4, HER2, SDF-1a, MMP-9, ENA-78, leptin, RAGE, and IL-10 is also inhibited. According to aspects of the disclosure, the cancer is prostate cancer. According to aspects of the disclosure, the cancer is castration resistant prostate cancer.

According to aspects of the disclosure, methods of treatment of a subject having, or suspected of having, cancer includes administration of an effective amount of at least one inhibitor, thereby inhibiting two or more of: CXCR4, IL-8, IL-6, HER2, TNFα, IL-1β, SDF-1a, MMP-9, ENA-78, leptin, RAGE, and IL-10 in the subject, with the proviso that, when CXCR4 and TNFα are inhibited, at least one additional member of the group: IL-8, IL-6, HER2, IL-1β, SDF-1a, MMP-9, ENA-78, leptin, RAGE, and IL-10 is also inhibited; with the proviso that, when CXCR4 and SDF-1a are inhibited, at least one additional member of the group: IL-8, IL-6, HER2, IL-1β, TNFα, MMP-9, ENA-78, leptin, RAGE, and IL-10 is also inhibited; with the proviso that, when IL-8, IL-6, and TNFα are inhibited, at least one additional member of the group: CXCR4, HER2, SDF-1a, MMP-9, ENA-78, leptin, RAGE, and IL-10 is also inhibited; and with the proviso that when IL-8, IL-6, TNFα, and IL-1β are inhibited, at least one additional member of the group: CXCR4, HER2, SDF-1a, MMP-9, ENA-78, leptin, RAGE, and IL-10 is also inhibited. According to aspects of the disclosure, the cancer is prostate cancer. According to aspects of the disclosure, the cancer is castration resistant prostate cancer.

Methods of treatment of a subject having, or suspected of having, cancer are provided according to aspects of the present disclosure which include: inhibiting two or more of: IL-8, IL-6, HER2, TNFα, IL-1β, SDF-1a, MMP-9, ENA-78, leptin, RAGE, and IL-10 in the subject, with the proviso that, when IL-8, IL-6, and TNFα are inhibited, at least one additional member of the group: HER2, SDF-1a, MMP-9, ENA-78, leptin, RAGE, and IL-10 is also inhibited; and with the proviso that when IL-8, IL-6, TNFα, and IL-1β are inhibited, at least one additional member of the group: HER2, SDF-1a, MMP-9, ENA-78, leptin, RAGE, and IL-10 is also inhibited. According to aspects of the disclosure, methods of treatment of a subject having, or suspected of having, cancer includes administration of an effective amount of at least one inhibitor, thereby inhibiting two or more of: IL-8, IL-6, HER2, TNFα, IL-1β, SDF-1a, MMP-9, ENA-78, leptin, RAGE, and IL-10 in the subject, with the proviso that, when IL-8, IL-6, and TNFα are inhibited, at least one additional member of the group: HER2, SDF-1a, MMP-9, ENA-78, leptin, RAGE, and IL-10 is also inhibited; and with the proviso that when IL-8, IL-6, TNFα, and IL-1β are inhibited, at least one additional member of the group: HER2, SDF-1a, MMP-9, ENA-78, leptin, RAGE, and IL-10 is also inhibited. According to aspects of the disclosure, the cancer is prostate cancer. According to aspects of the disclosure, the cancer is castration resistant prostate cancer.

Methods of treatment of a subject having, or suspected of having, cancer are provided according to aspects of the present disclosure which include: inhibiting two or more of: IL-8, IL-6, TNFα, ENA-78, leptin, RAGE, and IL-10 in the subject, with the proviso that, when IL-8, IL-6, and TNFα are inhibited, at least one additional member of the group: ENA-78, leptin, RAGE, and IL-10 is also inhibited. According to aspects of the disclosure, methods of treatment of a subject having, or suspected of having, cancer includes administration of an effective amount of at least one inhibitor, thereby inhibiting two or more of: IL-8, IL-6, TNFα, ENA-78, leptin, RAGE, and IL-10 in the subject, with the proviso that, when IL-8, IL-6, and TNFα are inhibited, at least one additional member of the group: ENA-78, leptin, RAGE, and IL-10 is also inhibited. According to aspects of the disclosure, the cancer is prostate cancer. According to aspects of the disclosure, the cancer is castration resistant prostate cancer.

According to aspects of the disclosure, the inhibitor or inhibitors, can be, or can include, a nucleic acid inhibitor, an antibody, an antigen-binding antibody fragment, an aptamer, an inorganic or organic small molecule inhibitor, or a combination of any two or more thereof.

According to aspects of the disclosure, administration of one or more inhibitors can be a systemic administration, a local administration, or both systemic and local, such as simultaneous systemic administrations, sequential systemic administrations, simultaneous local administrations, sequential local administrations, simultaneous systemic and local administrations, or sequential systemic and local administrations.

According to aspects of the disclosure, methods of treatment of a subject having, or suspected of having, cancer include assaying one or more of: CXCR4, IL-8, IL-6, HER2, TNFα, IL-1β, SDF-1a, MMP-9, ENA-78, leptin, RAGE, and IL-10 in a subject sample.

According to aspects of the disclosure, methods of treatment of a subject having, or suspected of having, cancer include assaying one or more of CXCR4, IL-8, IL-6, HER2, TNFα, IL-1β, SDF-1a, MMP-9, ENA-78, leptin, RAGE, and IL-10 in a subject sample obtained from the subject prior to treatment inhibiting two or more of: CXCR4, IL-8, IL-6, HER2, TNFα, IL-1β, SDF-1a, MMP-9, ENA-78, leptin, RAGE, and IL-10 in the subject.

According to aspects of the disclosure, methods of treatment of a subject having, or suspected of having, cancer include assaying one or more of CXCR4, IL-8, IL-6, HER2, TNFα, IL-1β, SDF-1a, MMP-9, ENA-78, leptin, RAGE, and IL-10 in a subject sample produces an assay result determining that one or more of: CXCR4, IL-8, IL-6, HER2, TNFα, IL-1β, SDF-1a, MMP-9, ENA-78, leptin, RAGE, and IL-10 is dysregulated in the subject.

According to aspects of the disclosure, methods of treatment of a subject having, or suspected of having, cancer include assaying comprises assaying one or more of CXCR4, IL-8, IL-6, HER2, TNFα, IL-1β, SDF-1a, MMP-9, ENA-78, leptin, RAGE, and IL-10 in a subject sample obtained from the subject following treatment inhibiting two or more of: CXCR4, IL-8, IL-6, HER2, TNFα, IL-1β, SDF-1a, MMP-9, ENA-78, leptin, RAGE, and IL-10 in the subject.

According to aspects of the disclosure, methods of treatment of a subject having, or suspected of having, cancer include assaying at least one of: ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3 to determine an aggressive or non-aggressive phenotype of the cancer.

According to aspects of the disclosure, methods of treatment of a subject having, or suspected of having, cancer include assaying at least one of: ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3 to determine an aggressive or non-aggressive phenotype of the cancer.

According to aspects of the disclosure, methods of treatment of a subject having, or suspected of having, cancer include assaying at least one of: ADIPOQ, AKT1, ALOX12, ALOX15, BMP2, CGA, CXCR4, CYP19A1, ERG, FASN, IL1B, IL6, IL8, NFKB1, PIK3C3, PIK3CA, PIK3R1, PLA2G2A, TGFB1, MMP-9, STAT3 and TNF-α to determine an aggressive or non-aggressive phenotype of the cancer.

According to aspects of the disclosure, methods of treatment of a subject having, or suspected of having, cancer include assaying at least one of: ADIPOQ, AKT1, ALOX12, ALOX15, BMP2, CGA, CXCR4, CYP19A1, ERG, FASN, IL1B, IL6, IL8, NFKB1, PIK3C3, PIK3CA, PIK3R1, PLA2G2A, TGFB1, MMP-9, STAT3 and TNF-α to determine an aggressive or non-aggressive phenotype of the cancer.

Method of assessing aggressiveness of a cancer in a subject are provided according to aspects of the present disclosure which include assaying one or more pro-inflammatory factors selected from the group consisting of: CXCR4, IL-8, IL-6, HER2, TNFα, IL-1β, SDF-1a, MMP-9, ENA-78, leptin, RAGE, and IL-10 in a subject sample; and comparing a result of the assaying to a control or standard, wherein when at least one, at least two, at least three, at least four, at least five, at least six, at least seven, at least eight, at least nine, at least ten, at least eleven, or all of the pro-inflammatory factors are determined to be dysregulated, the cancer is determined to be more likely to be aggressive.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols, 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; Herdewijn, P. (Ed.), Oligonucleotide Synthesis: Methods and Applications, Methods in Molecular Biology, Humana Press, 2004; Harlow, E. and Lane, D., Antibodies: A Laboratory Manual, Cold Spring Harbor Laboratory Press, 1988; Ausubel. F. et al., (Eds.), Short Protocols in Molecular Biology, Wiley, 2002; J. D. Pound (Ed.) Immunochemical Protocols, Methods in Molecular Biology, Humana Press, 2nd ed., 1998; B. K. C. Lo (Ed.); Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

Methods of treatment of cancer in a subject in need thereof are provided according to aspects of the present disclosure which include administering an effective amount of an inhibitor of a pro-inflammatory factor.

The term “effective amount” as used herein refers to an amount needed to elicit the desired biological response in the subject being treated. In subjects having cancer or at risk for having cancer, an effective amount is effective to ameliorate one or more signs and/or symptoms of the condition.

The term “a pro-inflammatory factor” as used herein refers to one or more of a pro-inflammatory cytokine, a pro-inflammatory chemokine, a pro-inflammatory cytokine receptor, a pro-inflammatory chemokine receptor, or a stimulator of any of these. According to particular aspects a pro-inflammatory factor to be inhibited in a subject in a method of treatment disclosed herein is dysregulated in the subject, and includes one or more of: a dysregulated pro-inflammatory cytokine, a dysregulated pro-inflammatory chemokine, a dysregulated pro-inflammatory cytokine receptor, a dysregulated pro-inflammatory chemokine receptor, or a dysregulated stimulator of any of these.

Methods of treatment of cancer in a subject in need thereof are provided according to aspects of the present disclosure which include administering an effective amount of an inhibitor of a pro-inflammatory factor to the subject, wherein the inhibitor is an inhibitor of a pro-inflammatory factor selected from the group consisting of: interleukin-1β (IL-1β), interleukin-6 (IL-6), interleukin-8 (IL-8), interleukin-10 (IL-10), stromal cell-derived factor-1α (SDF-1α, also known as C-X-C motif chemokine ligand 12 (CXCL12)), matrix metallopeptidase 9 (MMP-9)), epithelial-derived neutrophil-activating peptide 78 (ENA-78, also known as C-X-C motif chemokine ligand 5 (CXCL5)), receptor for advanced glycation end products (RAGE), TNF-α (tumor necrosis factor-α), HER2 (human epidermal growth factor receptor 2, also known as receptor tyrosine-protein kinase erbB2 or CD340), CXCR4 (C-X-C motif chemokine receptor 4), and leptin.

Methods of treatment of cancer in a subject in need thereof are provided according to aspects of the present disclosure which include administering an effective amount of an inhibitor of a pro-inflammatory factor to the subject, wherein the pro-inflammatory factor is dysregulated in the subject, and wherein the inhibitor is an inhibitor of a pro-inflammatory factor selected from the group consisting of: interleukin-1β (IL-1β), interleukin-6 (IL-6), interleukin-8 (IL-8), interleukin-10 (IL-10), stromal cell-derived factor-1α (SDF-1α, also known as C-X-C motif chemokine ligand 12 (CXCL12)), matrix metallopeptidase 9 (MMP-9), epithelial-derived neutrophil-activating peptide 78 (ENA-78, also known as C-X-C motif chemokine ligand 5 (CXCL5)), receptor for advanced glycation end products (RAGE), TNF-α (tumor necrosis factor-α), HER2 (human epidermal growth factor receptor 2, also known as receptor tyrosine-protein kinase erbB2 or CD340), CXCR4 (C-X-C motif chemokine receptor 4), and leptin.

The terms “subject” and “patient” are used interchangeably herein.

A subject treated according to methods and using compositions of the present disclosure can be mammalian or non-mammalian. A mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. A non-mammalian subject can be any non-mammal including, but not limited to, a bird such as a duck, goose, chicken, or turkey.

A subject can be either gender and can be any age.

According to particular aspects, the subject is human. In certain aspects, the subject is a human male of African descent (AAM) or a human male of European descent (EAM).

According to particular aspects, the subject has cancer, or is suspected of having cancer. Cancers treated using methods and compositions described herein are characterized by abnormal cell proliferation including, but not limited to, pre-neoplastic hyperproliferation, cancer in-situ, neoplasms, and metastasis, and include solid and non-solid tumors. Examples of cancers treated according to aspects of the present disclosure include, but are not limited to, lymphoma, leukemia, squamous cell cancer, small-cell lung cancer, non-small cell lung cancer, adenocarcinoma of the lung, squamous carcinoma of the lung, cancer of the peritoneum, adrenal cancer, anal cancer, bile duct cancer, bladder cancer, brain cancer, breast cancer, triple negative breast cancer, central or peripheral nervous system cancers, cervical cancer, colon cancer, colorectal cancer, endometrial cancer, esophageal cancer, gall bladder cancer, gastrointestinal cancer, glioblastoma, head and neck cancer, kidney cancer, liver cancer, nasopharyngeal cancer, nasal cavity cancer, oropharyngeal cancer, oral cavity cancer, osteosarcoma, ovarian cancer, pancreatic cancer, parathyroid cancer, pituitary cancer, prostate cancer, retinoblastoma, sarcoma, salivary gland cancer, skin cancer, small intestine cancer, stomach cancer, testicular cancer, thymus cancer, thyroid cancer, uterine cancer, vaginal cancer and vulval cancer.

According to particular aspects, the subject has prostate cancer, or is suspected of having prostate cancer.

Cancers treated using methods and compositions according to aspects of the present disclosure are characterized by abnormal cell proliferation and a dysregulated cytokine, a dysregulated chemokine, a dysregulated cytokine receptor, a dysregulated chemokine receptor, a dysregulated stimulator of any of the forgoing, or a combination of any two or more thereof.

Cancers treated using methods and compositions according to aspects of the present disclosure are characterized by abnormal cell proliferation and a dysregulated pro-inflammatory factor which is a dysregulated cytokine, a dysregulated chemokine, a dysregulated cytokine receptor, a dysregulated chemokine receptor, or a dysregulated stimulator of any of the foregoing, selected from the group consisting of: IL-1β, IL-6, IL-8, IL-10, SDF-1α, MMP-9, ENA-78, RAGE, TNF-α, HER2, CXCR4, leptin, and any two, three, four, five, six, seven, eight, nine, ten, eleven, or all thereof.

According to particular aspects, the subject has prostate cancer, or is suspected of having prostate cancer, including pre-neoplastic hyperproliferation, cancer in-situ, neoplasms, and metastasis, and include solid and non-solid tumors. Prostate cancers treated using methods and compositions according to aspects of the present disclosure are characterized by abnormal cell proliferation and a dysregulated pro-inflammatory factor which is a dysregulated cytokine, a dysregulated chemokine, a dysregulated cytokine receptor, a dysregulated chemokine receptor, a dysregulated stimulator of any of the foregoing, or any two or more thereof.

Prostate cancers treated using methods and compositions according to aspects of the present disclosure are characterized by abnormal cell proliferation, and a dysregulated pro-inflammatory factor which is a dysregulated cytokine, a dysregulated chemokine, a dysregulated cytokine receptor, a dysregulated chemokine receptor, a dysregulated stimulator of any of the foregoing, or any two or more thereof, selected from the group consisting of: IL-1β, IL-6, IL-8, IL-1β, SDF-1a, MMP-9, ENA-78, RAGE, TNF-α, HER2, CXCR4, leptin, and any two or more thereof.

Prostate cancers treated using methods and compositions according to aspects of the present disclosure are characterized by abnormal cell proliferation, and three, four, five, six, seven, eight, nine, ten, eleven, or all of: dysregulated IL-1β, dysregulated IL-6, dysregulated IL-8, dysregulated IL-1β, dysregulated SDF-1α, dysregulated MMP-9, dysregulated ENA-78, dysregulated RAGE, dysregulated TNF-α, dysregulated HER2, dysregulated CXCR4, and dysregulated leptin.

Methods and compositions of the present disclosure can be used for prophylaxis as well as amelioration of signs and/or symptoms of cancer. The terms “treating” and “treatment” used to refer to treatment of a cancer in a subject include: preventing, inhibiting or ameliorating the cancer in the subject, such as slowing progression of the cancer and/or reducing or ameliorating a sign or symptom of the cancer.

Methods and compositions of the present disclosure can be used for prophylaxis as well as amelioration of signs and/or symptoms of prostate cancer. The terms “treating” and “treatment” used to refer to treatment of a prostate cancer in a subject include: preventing, inhibiting or ameliorating the prostate cancer in the subject, such as slowing progression of the prostate cancer and/or reducing or ameliorating a sign or symptom of the prostate cancer.

According to aspects, a subject is an individual in need of diagnosis based on particular symptoms or family history.

The term “dysregulated” as used herein to refer to a pro-inflammatory factor, particularly a pro-inflammatory cytokine, pro-inflammatory chemokine, pro-inflammatory cytokine receptor, pro-inflammatory chemokine receptor, or stimulator of any of the foregoing, indicates that the expression, level, activity, localization, or other characteristic of the cytokine, chemokine, cytokine receptor, or chemokine receptor, is abnormal in a subject sample compared to a control.

According to particular aspects of the present disclosure, a pro-inflammatory factor, particularly a pro-inflammatory cytokine, pro-inflammatory chemokine, pro-inflammatory cytokine receptor, pro-inflammatory chemokine receptor, or stimulator of any of the foregoing, is determined to be dysregulated in a subject having cancer or suspected of having cancer, by an assay to assess the expression, level, activity, localization, or other characteristic of one or more of: a pro-inflammatory cytokine, pro-inflammatory chemokine, pro-inflammatory cytokine receptor, pro-inflammatory chemokine receptor, or stimulator of any of the foregoing, in a subject sample.

According to particular aspects of the present disclosure, a pro-inflammatory factor, particularly a pro-inflammatory cytokine, pro-inflammatory chemokine, pro-inflammatory cytokine receptor, pro-inflammatory chemokine receptor, or stimulator of any of the foregoing, is determined to be dysregulated in a subject having cancer, or suspected of having cancer, by an assay to assess the expression, level, activity, localization, or other characteristic of one, two, three, four, five, six, seven, eight, nine, ten, eleven, or all of: IL-1β, IL-6, IL-8, IL-10, SDF-1α, MMP-9, ENA-78, RAGE, TNF-α, HER2, CXCR4, and leptin, in a subject sample.

According to particular aspects of the present disclosure, a pro-inflammatory factor, particularly a pro-inflammatory cytokine, pro-inflammatory chemokine, pro-inflammatory cytokine receptor, pro-inflammatory chemokine receptor, or stimulator of any of the foregoing, is determined to be dysregulated in a subject having prostate cancer or suspected of having prostate cancer, by an assay to assess the expression, level, activity, localization, or other characteristic of a pro-inflammatory factor, particularly a pro-inflammatory cytokine, pro-inflammatory chemokine, pro-inflammatory cytokine receptor, pro-inflammatory chemokine receptor, or stimulator of any of the foregoing, in a subject sample.

According to particular aspects of the present disclosure, a pro-inflammatory factor, particularly a pro-inflammatory cytokine, pro-inflammatory chemokine, pro-inflammatory cytokine receptor, pro-inflammatory chemokine receptor, or stimulator of any of the foregoing, is determined to be dysregulated in a subject having prostate cancer, or suspected of having prostate cancer, by an assay to assess the expression, level, activity, localization, or other characteristic of one, two, three, four, five, six, seven, eight, nine, ten, eleven, or all of: IL-1β, IL-6, IL-8, IL-10, SDF-1a, MMP-9, ENA-78, RAGE, TNF-α, HER2, CXCR4, and leptin, in a subject sample.

The term “subject sample” as used herein refers to any material, including, without limitation, a biological fluid, cell or tissue, obtained from, or derived from, a subject that contains, or is suspected of containing, the substance to be assayed, illustratively including blood, plasma, serum, urine, saliva, ascites, cerebrospinal fluid, cerebroventricular fluid, pleural fluids, pulmonary and bronchial lavage samples, mucous, sweat, tears, semen, bladder wash samples, amniotic fluid, lymph, peritoneal fluid, synovial fluid, bone marrow aspirate, tumor cells or tissue, organ cells or tissue, such as biopsy material. According to aspects of the present disclosure, a subject sample includes cultured cells or cultured tissues. According to aspects of the present disclosure, a subject sample includes cells or tissues implanted in an animal of a different species, such as human cells or tissues implanted in a mouse.

According to aspects of the present disclosure, a “subject sample” is a blood sample obtained from a subject having, or suspected of having prostate cancer. According to aspects of the present disclosure, a “subject sample” is a plasma sample obtained from a subject having, or suspected of having prostate cancer. According to aspects of the present disclosure, a “subject sample” is a prostate tissue sample obtained from a subject having, or suspected of having prostate cancer. According to aspects of the present disclosure, a “subject sample” is a semen or urine sample obtained from a subject having, or suspected of having prostate cancer.

An assay to assess dysregulation of one or more of: a pro-inflammatory factor, particularly a pro-inflammatory cytokine, pro-inflammatory chemokine, pro-inflammatory cytokine receptor, pro-inflammatory chemokine receptor, or stimulator of any of the foregoing, in a subject sample, are performed using techniques such as nucleic acid assays, spectrometric assays, immunoassays, and functional assays. A mutation status of one or more of: a pro-inflammatory factor, particularly a pro-inflammatory cytokine, pro-inflammatory chemokine, pro-inflammatory cytokine receptor, pro-inflammatory chemokine receptor, or stimulator of any of the foregoing, in a subject sample, can be assessed using assays such as protein or peptide sequencing, nucleic acid assay and immunoassay.

Nucleic acid assays for assessing nucleic acids encoding a pro-inflammatory factor, particularly a pro-inflammatory cytokine, pro-inflammatory chemokine, pro-inflammatory cytokine receptor, pro-inflammatory chemokine receptor, or stimulator of any of the foregoing, particularly mRNA or cDNA, in a subject sample, include, but are not limited to, sequencing; polymerase chain reactions (PCR) such as RT-PCR; dot blot; in situ hybridization; Northern blot; RNase protection; RNase protection assay; microarray analysis; and cDNA-mediated Annealing, Selection, extension, and Ligation (DASL) assay (Chow et al., Front Genet. 2012, 3:11). Nucleic acid assays are described in detail, for example, in Sambrook, J. and Russell, D. W., Molecular Cloning: A Laboratory Manual, Cold Spring Harbor Laboratory Press, 3rd Ed, 2001; and F. M. Ausubel, Ed., Short Protocols in Molecular Biology, Current Protocols, 5th Ed., 2002.

Immunoassays can be used to assess a pro-inflammatory factor, particularly a pro-inflammatory cytokine, pro-inflammatory chemokine, pro-inflammatory cytokine receptor, pro-inflammatory chemokine receptor, or stimulator of any of the foregoing, in a subject sample, including, but not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay (LIA), fluorescent immunoassay (FIA), and radioimmunoassay. Immunoassays are described in detail, for example, in Harlow, E. and Lane, D., Antibodies: A Laboratory Manual, Cold Spring Harbor Laboratory Press, 1988; Ausubel. F. et al., (Eds.), Short Protocols in Molecular Biology, Wiley, 2002; and J. D. Pound (Ed.) Immunochemical Protocols, Methods in Molecular Biology, Humana Press, 2nd ed., 1998; B. K. C. Lo (Ed.).

Functional assays can be used to assess a pro-inflammatory factor, particularly a pro-inflammatory cytokine, pro-inflammatory chemokine, pro-inflammatory cytokine receptor, pro-inflammatory chemokine receptor, or stimulator of any of the foregoing, in a subject sample, including, but not limited to, assessing activity of the cytokine, the chemokine, the cytokine receptor, or the chemokine receptor to bind to a binding partner, such as a ligand or receptor.

Spectrometric analysis can be used to assay a sample for one or more pro-inflammatory factors of the present disclosure. Any of various spectroscopy methods can be used to assay one or more pro-inflammatory factors and/or one or more biomarkers of the present disclosure, including, but not limited to, gas chromatography, liquid chromatography, ion mobility spectrometry, mass spectrometry, liquid chromatography-mass spectrometry (LC-MS or HPLC-MS), ion mobility spectrometry-mass spectrometry, tandem mass spectrometry, gas chromatography-mass spectrometry, matrix-assisted desorption ionization time-of-flight (MALDI-TOF) mass spectrometry, surface-enhanced laser desorption ionization (SELDI) and nuclear magnetic resonance spectroscopy, all of which are well-known to the skill artisan.

For example, mass analysis can be used in an assay according to aspects of the present disclosure. Mass analysis is conducted using, for example, time-of-flight (TOF) mass spectrometry or Fourier transform ion cyclotron resonance mass spectrometry. Mass spectrometry techniques are known in the art and exemplary detailed descriptions of methods for protein and/or peptide assay are found in Li J., et al., Clin Chem., 48(8):1296-304, 2002; Hortin, G. L., Clinical Chemistry 52: 1223-1237, 2006; Hortin, G. L., Clinical Chemistry 52: 1223-1237, 2006; A. L. Burlingame, et al. (Eds.), Mass Spectrometry in Biology and Medicine, Humana Press, 2000; and D. M. Desiderio, Mass Spectrometry of Peptides, CRC Press, 1990.

Assays according to aspects of the present disclosure can be multiplex, i.e. assays for two or more pro-inflammatory factors.

One or more standards and/or controls can be used to allow assessment, including quantitative assessment, of gene, biomarker, and/or a pro-inflammatory factor.

One or more standards and/or controls can be used to allow assessment, of dysregulation of a pro-inflammatory factor, particularly a pro-inflammatory cytokine, pro-inflammatory chemokine, pro-inflammatory cytokine receptor, pro-inflammatory chemokine receptor, or stimulator of any of the foregoing, in a subject sample.

According to aspects of the present disclosure, assaying a pro-inflammatory factor, particularly a pro-inflammatory cytokine, pro-inflammatory chemokine, pro-inflammatory cytokine receptor, pro-inflammatory chemokine receptor, or stimulator of any of the foregoing, to detect dysregulation thereof includes use of a control, also referred to interchangeably as a “suitable control,” an “appropriate control,” a “control sample,” or a “reference.” Thus, assays according to aspects of the present disclosure may include a step that involves comparing a detected aspect, such as level, activity, localization, or other characteristic, to a suitable control. A control is any control or standard useful for comparison purposes to detect dysregulation of a pro-inflammatory factor, particularly a pro-inflammatory cytokine, pro-inflammatory chemokine, pro-inflammatory cytokine receptor, pro-inflammatory chemokine receptor, or stimulator of any of the foregoing, in a subject sample from a subject having or suspected of having cancer. In some aspects, a control is a level, activity, localization, expression characteristic, or other characteristic of a pro-inflammatory factor, particularly a pro-inflammatory cytokine, pro-inflammatory chemokine, pro-inflammatory cytokine receptor, pro-inflammatory chemokine receptor, or stimulator of any of the foregoing, determined in a subject, or population of, “normal” subjects. A control can be a reference value previously determined and stored in a print or electronic medium for recall and comparison.

In other aspects, a control is a detected aspect, such as expression, level, activity, localization, or other characteristic, of a pro-inflammatory factor, particularly a pro-inflammatory cytokine, pro-inflammatory chemokine, pro-inflammatory cytokine receptor, pro-inflammatory chemokine receptor, or stimulator of any of the foregoing, determined prior to treating the subject having, or suspected of having, cancer, which is compared to a detected aspect, such as expression, level, activity, localization, or other characteristic, of a pro-inflammatory factor, particularly a pro-inflammatory cytokine, pro-inflammatory chemokine, pro-inflammatory cytokine receptor, pro-inflammatory chemokine receptor, or stimulator of any of the foregoing, determined during or after treating the subject.

A control can be a profile or pattern of one or more aspects detected by an assay, such as expression, level, activity, localization, or other characteristic, of one or more pro-inflammatory factors which can be compared with a profile or pattern of one or more aspects determined in a subject sample.

A control can be an amount of one or more pro-inflammatory factors of the present disclosure present in a comparable sample obtained from the same subject at a different time. For example, a standard can be an amount of one or more pro-inflammatory factors of the present disclosure present in a comparable sample obtained from the same subject at an earlier time. A first sample can be obtained from an individual subject at a first time to obtain a subject-specific baseline level of the one or more pro-inflammatory factors of the present disclosure in the first sample. A second sample can be obtained from the individual subject at a second time and assayed for one or more pro-inflammatory factors of the present disclosure to monitor differences in the levels of the one or more pro-inflammatory factors of the present disclosure compared to the first sample, thereby monitoring health and/or disease in the subject. Additional samples can be obtained from the subject at additional time points and assayed for pro-inflammatory factors to monitor differences in the levels of the corresponding one or more pro-inflammatory factors of the present disclosure compared to the first sample, second sample or other samples, thereby monitoring health and/or disease in the subject.

A standard can be an average level of one or more pro-inflammatory factors of the present disclosure present in comparable samples of one or more populations. The “average level” is determined by assay of the one or more pro-inflammatory factors of the present disclosure in comparable samples obtained from each member of the population. The term “comparable sample” is used to indicate that the samples are of the same type. For example, each of the comparable samples is a plasma sample. In a further example, each of the comparable samples is a biopsy sample.

A difference detected in levels of one or more pro-inflammatory factors of the present disclosure compared to a standard can be an increase or decrease in level of the one or more pro-inflammatory factors. A difference detected in levels of one or more pro-inflammatory factors of the present disclosure compared to a standard can be a detectable level of the one or more pro-inflammatory factors where the pro-inflammatory factor is undetectable in the standard. A difference detected in levels of one or more pro-inflammatory factors of the present disclosure compared to a standard can be an undetectable level of the one or more pro-inflammatory factors where the biomarker is detectable in the standard.

Assay results can be analyzed using statistical analysis by any of various methods, exemplified by parametric or non-parametric tests, analysis of variance, analysis of covariance, logistic regression for multivariate analysis, Fisher's exact test, the chi-square test, Student's T-test, the Mann-Whitney test, Wilcoxon signed ranks test, McNemar test, Friedman test and Page's L trend test. These and other statistical tests are well-known in the art as detailed in Hicks, C M, Research Methods for Clinical Therapists: Applied Project Design and Analysis, Churchill Livingstone; 5th Ed., 2009; and Freund, R J et al., Statistical Methods, Academic Press; 3rd Ed., 2010.

Proinflammatory Marker Score

According to aspects of the present disclosure, determination that one, two, three, four, five, six, seven, eight, nine, ten, eleven, or all, of IL-1β, IL-6, IL-8, IL-10, ENA-78, leptin, RAGE, SDF-1a, TNF-α, HER2, CXCR4, and MMP-9 are dysregulated indicates a more highly aggressive cancer in the subject. As the number of dysregulated indicators selected from IL-1β, IL-6, IL-8, IL-10, ENA-78, leptin, RAGE, SDF-1a, TNF-α, HER2, CXCR4, and MMP-9, increases, the cancer in the subject is determined to be more aggressive.

According to aspects of the present disclosure, results of assays for pro-inflammatory markers are considered to determine a “proinflammatory marker score.” The proinflammatory marker score is based on the cumulative number of pro-inflammatory markers determined to be dysregulated. A higher proinflammatory marker score is indicative of more aggressive cancer and conversely a lower proinflammatory marker score is indicative of less aggressive cancer.

According to aspects of the present disclosure, results of assays for one, two, three, four, five, six, seven, eight, nine, ten, eleven, or all, of IL-1β, IL-6, IL-8, IL-10, ENA-78, leptin, RAGE, SDF-1a, TNF-α, HER2, CXCR4, and MMP-9 are considered to determine a proinflammatory marker score.

According to aspects of the present disclosure, plasma levels of one, two, three, four, five, six, seven, eight, nine, ten, eleven, or all, of IL-1β, IL-6, IL-8, IL-10, ENA-78, leptin, RAGE, SDF-1a, TNF-α, HER2, CXCR4, and MMP-9 are measured by an assay or one or more plasma samples of a subject, to determine a proinflammatory marker score.

According to aspects of the present disclosure, detection of dysregulation of particular pro-inflammatory factors, including pro-inflammatory cytokines, pro-inflammatory chemokines, pro-inflammatory cytokine receptors, pro-inflammatory chemokine receptors, pro-inflammatory stimulators of any of the foregoing, or combinations thereof, is indicative of aggressive prostate cancer in AAM.

According to aspects of the present disclosure, detection of dysregulation of one or more, two or more, three or more, or all four of: ENA-78, RAGE, leptin, and IL-10 is indicative of aggressive prostate cancer in AAM.

Gene Score

According to aspects of the present disclosure, one or more functionally related, prostate cancer driver genes are assayed in a subject having, or suspected of having, prostate cancer, wherein the one or more functionally related, prostate cancer driver genes is selected from the group consisting of: Adiponectin (ADIPOQ); Rac Protein Kinase Alpha (AKT-1); Arachidonate 12-Lipoxygenase (ALOX12); Arachidonate 15-Lipoxygenase (ALOX15); Arachidonate 15-Lipoxygenase, Type B (ALOX15B); Bone Morphogenetic Protein 2 (BMP2); Chorionic Gonadotrophin Subunit Alpha (CGA); C-X-C chemokine receptor type 4 (CXCR4); Cytochrome P450, Family 19, Subfamily A, Polypeptide 1 (CYP19A1); ETS Related Gene (ERG); Fatty Acid Synthase (FASN); Interleukin-1 beta (ILB1); Interleukin 6 (IL6); Interleukin 8 (IL8); Nuclear Factor Of Kappa Light Polypeptide Gene Enhancer In B-Cells 1 (NFKB1); Phosphatidylinositol 3-Kinase, Catalytic Subunit Type 3 (PIK3C3); Phosphatidylinositol-4,5-Bisphosphate 3-Kinase, Catalytic Subunit Alpha (PIK3CA); Phosphoinositide-3-Kinase, Regulatory Subunit 1 (PIK3R1); Phospholipase A2, Group IIA (PLA2G2A); Transforming Growth Factor, Beta 1 (TGFB1); Tissue Inhibitor Of Metalloproteinases 3 (TIMP3), or any combination thereof to generate a “gene score.” These genes or biomarkers can be differentially present/expressed in non-aggressive prostate cancer and aggressive prostate cancer as well as in males of either African or European descent, and are therefore useful in aiding in the accurate determination of aggressive prostate cancer status in these two separate patient cohorts.

According to aspects of the present disclosure, one or more functionally related, prostate cancer driver genes are assayed in a subject having, or suspected of having, prostate cancer to generate a gene score, wherein the one or more functionally related, prostate cancer driver genes is selected from the group consisting of: MMP-9, STAT3 and/or TNF-α. Any one or more of these may be assayed in addition to, or instead of, any one or more of ADIPOQ; AKT-1; ALOX12; ALOX15; ALOX15B; BMP2; CGA; CXCR4; CYP19A1; ERG; FASN; ILB1; IL6; IL8; NFKB1; PIK3C3; PIK3CA; PIK3R1; PLA2G2A; TGFB1; TIMP3, or a combination of any two or more thereof.

The functionally related, prostate cancer driver genes and biomarkers provided above can be differentially expressed or present in non-aggressive prostate cancer or aggressive prostate cancer differentiating by the number of genes above and below a determined expression threshold number in males of either African or European descent, and, therefore, are useful in aiding in the accurate determination of aggressive prostate cancer status in these two separate patient cohorts. In certain aspects, these genes or biomarkers are measured in a patient sample using the methods described herein and compared, for example, to predefined genes or biomarkers expression thresholds and correlated to aggressive prostate cancer status. In certain aspects, the expression threshold number for each of the twenty-one functionally related, prostate cancer driver genes or biomarkers disclosed herein by race (males of African descent v. males of European descent) is specific for the assay used to measure the gene or biomarker levels/ratios. In aspects, the expression threshold for each of the twenty-one functionally related, prostate cancer driver genes or biomarkers can be determined, for example, by measuring the amount or expression of these genes or biomarkers in a statistically significant number of samples from patients with the different aggressive prostate cancer statuses, and utilizing a defined aggressive phenotype and non-aggressive phenotype of prostate cancer in predicting prostate cancer disease aggressiveness using recursive partitioning. This results in an expression threshold number for each gene for each race, and can be used to define a high-risk and a low-risk subset for each gene for each race. The expression threshold for each of these genes will be specific to the assay used to measure the amount or expression of these genes or biomarkers, such as, e.g., DASL, genome wide or targeted RNA sequencing using whole genome microarray or target resequencing, respectively. In particular aspects, an aggressive phenotype (aggressive PCa defined as, e.g., Gene Score (GS)≥8 or 7 (4+3), T3 disease and BCR within 3 years) and a non-aggressive phenotype (non-aggressive PCa defined as, e.g., GS≤6, T2 disease, and no BCR within 5 years), can be used to identify the expression threshold for each of the twenty-one functionally related, prostate cancer driver genes, and/or the additional three, MMP-9, STAT3 and TNF-α, individually by race (males of African descent v. males of European descent) in predicting prostate cancer disease aggressiveness using recursive partitioning. In aspects, at least 30% of the sample is required to be in both of the daughter nodes. Therefore, for each race/gene combination, there will be a daughter node with a greater proportion of aggressive disease patients than the proportion from all samples. We denote this as the aggressive sub group.

Using the methods disclosed herein, the present inventors determined that for males of African descent, higher expression levels of ALOX15, BMP2, FASN, PIK3R1, PLA2G2A, and TGFB1 were associated with a more aggressive phenotype; while lower levels of ADIPOQ, AKT1, ALOX12, ALOX15B, CGA, CXCR4, CYP19A1, ERG, IL1B, IL6, IL8, NFKB1, PIK3C3, PIK3CA, and TIMP3 were associated with a more aggressive phenotype. Therefore, in some aspects, if the genes or biomarkers comprising ADIPOQ, AKT1, ALOX12, ALOX15, BMP2, CGA, CXCR4, CYP19A1, FASN, IL1B, IL8, NFKB1, PLA2G2A TGFB1, and TIMP3 are up-regulated in a subject of African descent compared to normalized expression values (e.g. normalized gene expression values in no cancer or non-aggressive prostate cancer patients), then a measured amount(s) above the expression threshold for each gene or biomarker provides a positive indication of aggressive prostate cancer. Alternatively, if the genes or biomarkers comprising ADIPOQ, AKT1, ALOX12, ALOX15, BMP2, CGA, CXCR4, CYP19A1, FASN, IL1B, IL8, NFKB1, PLA2G2A TGFB1, and TIMP3 are down-regulated in African American males compared to normalized expression values (e.g. normalized gene expression values in no cancer or non-aggressive prostate cancer patients), then a measured amount for each gene or biomarker below the expression threshold for each gene provides a negative indication of aggressive prostate cancer. Additionally, if the genes or biomarkers comprising ALOX15B, ERG, IL6, PIK3C3, PIK3CA, and PIK3R1, are down-regulated in males of African descent compared to normalized expression values (e.g., normalized gene expression values in no cancer or non-aggressive prostate cancer patients), then a measured amount for each gene or biomarker below the expression threshold provides a positive indication of aggressive prostate cancer. Alternatively, if the gene or biomarker comprising ALOX15B, ERG, IL6, PIK3C3, PIK3CA, and PIK3R1 are up-regulated in males of African descent compared to normalized expression values (e.g., normalized gene expression values in no cancer or non-aggressive prostate cancer patients), then a measured amount for each gene or biomarker above the expression threshold provides a negative indication of aggressive prostate cancer.

Using the methods disclosed herein, the present inventors determined that for males of European descent, higher expression levels of ADIPOQ, ALOX15, CGA CXCR4, CYP19A1, IL6, IL8, NFKB1, PIK3C3, PLA2G2A, TGFB1 and TIMP3 were associated with a more aggressive phenotype; while lower levels of AKT1, ALOX12, ALOX15B, BMP2, ERG, FASN, IL1B, PIK3CA and PIK3R1 were associated with a more aggressive phenotype. Therefore, in some aspects, if the genes or biomarkers comprising AKT1, ALOX12, ALOX15, CGA, CXCR4, CYP19A1, FASN, IL6, IL8, NFKB1, PIK3C3, PIK3CA, TGFB1, and TIMP3 are up-regulated in males of European descent compared to normalized expression values (e.g. normalized gene expression values in no cancer or non-aggressive prostate cancer patients), then a measured amount above the expression threshold for each gene or biomarker provides a positive indication of aggressive prostate cancer. Alternatively, if the genes or biomarkers comprising AKT1, ALOX12, ALOX15, CGA, CXCR4, CYP19A1, FASN, IL6, IL8, NFKB1, PIK3C3, PIK3CA, TGFB1, and TIMP3 are down-regulated in males of European descent compared to normalized expression values (e.g. normalized gene expression values in no cancer or non-aggressive prostate cancer patients), then a measured amounts below the expression threshold for each gene or biomarker provides a negative indication of aggressive prostate cancer. Additionally, if the genes or biomarkers comprising ADIPOQ, ALOX15B, BMP2, ERG, IL1B, PIK3R1, and PLA2G2A are down-regulated in males of European descent compared to normalized expression values (e.g. normalized gene expression values in no cancer or non-aggressive prostate cancer patients), then a measured amount below the optimal threshold for each gene or biomarker provides a positive indication of aggressive prostate cancer. Alternatively, if the genes or biomarkers comprising ADIPOQ, ALOX15B, BMP2, ERG, IL1B, PIK3R1, and PLA2G2A, are up-regulated in European American males compared to normalized expression values (e.g. normalized gene expression values in no cancer or non-aggressive prostate cancer patients), then a measured amount above the expression threshold for each gene or biomarker provides a negative indication of aggressive prostate cancer.

In aspects, the number of genes that a patient has that are high risk, based on comparison of the patient's gene expression compared to the expression threshold number for that particular gene, are summed to create a gene score. Recursive partitioning is again used to define a threshold for the gene score for each race, above which the patient has aggressive prostate cancer and below which the patient does not have aggressive prostate cancer. In aspects, at least 30% of the sample is required to be in both of the daughter nodes. Thus, in particular aspects, the number of genes that a patient has that are high risk may then be compared with a relevant diagnostic number of genes, cut-off(s), or multivariate model scores that distinguish a positive aggressive prostate cancer status from a negative aggressive prostate cancer status in males of African or European descent. The diagnostic number of genes represent a measured amount of the gene(s) or biomarker(s) above which or below which a patient is classified as having a particular aggressive prostate cancer status. As is well understood in the art, by adjusting the particular diagnostic cut-off(s) used in an assay, one can increase sensitivity or specificity of the diagnostic assay depending on the preference of the diagnostician. In particular aspects, the particular diagnostic cut-off can be determined, for example, by measuring the amount or expression of biomarkers in a statistically significant number of samples from patients with the different aggressive prostate cancer statuses, and drawing the cut-off to suit the desired levels of specificity and sensitivity. However, the desired cut off or threshold line to determine aggressive versus non-aggressive is that level that minimizes false positives and false negatives.

Additionally, in some aspects the risk of developing aggressive prostate cancer is determined by measuring the relevant genes or biomarkers and then either submitting them to a classification algorithm or comparing them with a reference amount, i.e., a predefined level or pattern of biomarkers that is associated with the particular risk level. In other aspects, the course of aggressive prostate cancer in a patient is determined. Aggressive prostate cancer course refers to changes in aggressive prostate cancer status over time. Over time, the amount or relative amount (e.g., the expression pattern or ratio) of the genes or biomarkers can change. Therefore, the trend of these genes or biomarkers may increase over time toward a more aggressive prostate cancer at different rates. Accordingly, this method involves measuring the level of these genes or biomarkers in a patient at different time points. The course of aggressive prostate cancer is determined based on these comparisons.

Therefore, in some aspects, a gene score is determined by adding up the positive indications of aggressive prostate cancer of the measured genes or biomarkers (e.g. comprising the twenty-one functionally related, prostate cancer driver genes or biomarkers) in a male patient of African descent (e.g. from an obtained biological sample from the patient). In some aspects, the expression level of the genes or biomarkers are measured by an appropriate assay, as described in more detail below. If the gene score for the patient is 11 or more (i.e. 11 or more of more positive indications of aggressive prostate cancer of the measured genes or biomarkers), then the patient, particularly males of African descent, is identified as likely having aggressive prostate cancer or a high risk of occurrence/recurrence of prostate cancer. In particular aspects, the resultant sensitivity and specificity of males of African descent with 11 or more positive indications of the twenty-one measured genes or biomarkers is 100% and 69%, respectively. In some aspects, if the patient is identified as likely having aggressive prostate cancer or a high risk of occurrence/recurrence of prostate cancer, then the method further comprises treating the patient with an appropriate therapeutic regimen for aggressive prostate cancer if the diagnosis of the patient correlates to aggressive prostate cancer. In other aspects, if the gene score for the patient is 10 or less (i.e. 10 or less of more positive indications of aggressive prostate cancer of the measured genes or biomarkers comprising the twenty one measured genes or biomarkers), then the patient is identified as likely having non-aggressive prostate cancer or a low risk of occurrence/recurrence of prostate cancer. In some aspects, if the patient is identified as likely having non-aggressive prostate cancer or a low risk of occurrence/recurrence of prostate cancer, then the method further comprises treating the patient with an appropriate therapeutic regimen for non-aggressive prostate cancer if the diagnosis of the patient correlates to non-aggressive prostate cancer.

In certain aspects, a gene score is determined by adding up the positive indications in the patient (e.g. from an obtained biological sample form the patient), particularly males of European descent. If the gene score for the patient is 10 or more (i.e. 10 or more positive indications of aggressive prostate cancer of the twenty-one measured genes or biomarkers), then the patient, particularly a male of European descent, is identified as likely having aggressive prostate cancer or a high risk of occurrence/recurrence of prostate cancer. In particular aspects, the resultant sensitivity and specificity of males of European descent with 10 or more positive indications of aggressive prostate cancer of the twenty one measured genes or biomarkers is 88% and 85%, respectively. In some aspects, if the patient is identified as likely having aggressive prostate cancer or a high risk of occurrence/recurrence of prostate cancer, then the method further comprises treating the patient with an appropriate therapeutic regimen for aggressive prostate cancer if the diagnosis of the patient correlates to aggressive prostate cancer. In other aspects, if the gene score for the patient is 9 or fewer (i.e. 9 or fewer of more positive indications of aggressive prostate cancer of the twenty one measured genes or biomarkers), then the patient is identified as likely having non-aggressive prostate cancer or a low risk of occurrence/recurrence of prostate cancer. In some aspects, if the patient is identified as likely having non-aggressive prostate cancer or a low risk of occurrence/recurrence of prostate cancer, then the method further comprises treating the patient with an appropriate therapeutic regimen for non-aggressive prostate cancer if the diagnosis of the patient correlates to non-aggressive prostate cancer.

As such, in aspects, a method for identifying a male of African descent as having or likely to have aggressive prostate cancer is provided. In some aspects, the method comprises the steps of: a) obtaining a biological sample from the patient; b) detecting expression levels in the biological sample of a group of prostate cancer driver genes, said genes comprising ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3; and c) determining the expression threshold level for each prostate cancer driver gene in step b. In aspects, determining the expression threshold level for each gene comprises: 1) measuring the amount or expression of each prostate cancer drive gene in step b or biomarkers in a statistically significant number of samples from male patients of African descent with the different aggressive prostate cancer statuses; and 2) utilizing a defined aggressive phenotype and non-aggressive phenotype of prostate cancer in predicting prostate cancer disease aggressiveness using recursive partitioning; d) determining a number of positive indications for aggressive prostate cancer by comparing the detected expression levels of each gene determined in step b) to the expression threshold level for each determined in step c) for each gene. The method further comprises the steps of: e) determining a gene score threshold using recursive partitioning; and f) identifying the patient as having or likely to have aggressive prostate cancer if there are more positive indications then the gene score threshold determined in step e). In certain aspects of a method for identifying a male of African descent as having or likely to have aggressive prostate cancer, the aggressive phenotype comprises a Gleason score of greater than or equal to 8 or 7 (4+3), T3 disease and BCR within 3 years. In aspects of method for identifying a male of African descent as having or likely to have aggressive prostate cancer the non-aggressive phenotype comprises a Gleason score of less than or equal to 6, T2 disease, and no BCR within 5 years.

In certain aspects of a method for identifying a male of African descent as having or likely to have aggressive prostate cancer, the recursive partitioning of step c) 2) comprises two daughter nodes. In some aspects, the daughter nodes of the recursive partitioning of step c) 2) require at least 30% of the sample to be in both daughter nodes. In certain aspects, the recursive partitioning step of step e) comprises two daughter nodes. In some aspects, the daughter nodes of the recursive partitioning of step e) require at least 30% of the sample to be in both daughter nodes.

In certain aspects of a method for identifying a male of African descent as having or likely to have aggressive prostate cancer, determining the number of positive indications comprises determining if ADIPOQ, AKT1, ALOX12, ALOX15, BMP2, CGA, CXCR4, CYP19A1, FASN, IL1B, IL8, NFKB1, PLA2G2A TGFB1, and TIMP3 are upregulated in the biological sample to a level greater than the expression threshold level for each gene and determining if ALOX15B, ERG, IL6, PIK3C3, PIK3CA, and PIK3R1 are down regulated in the biological sample to a level less than the expression threshold level for each gene.

In certain aspects of a method for identifying a male of African descent as having or likely to have aggressive prostate cancer, the method further comprises identifying the patient as having or likely to have nonaggressive prostate cancer if there are there are fewer positive indications then the gene score threshold determined in step e).

In certain aspects of a method for identifying a male of African descent as having or likely to have aggressive prostate cancer, the gene threshold score is eleven.

In certain aspects of a method for identifying a male of African descent as having or likely to have aggressive prostate cancer, wherein the biological sample is a tumor biopsy.

In certain aspects of a method for identifying a male of African descent as having or likely to have aggressive prostate cancer, the step of detecting comprises PCR, DASL, genome wide RNA sequencing, targeted RNA sequencing, or an immunoassay.

In certain aspects of a method for identifying a male of African descent as having or likely to have aggressive prostate cancer, the method further comprises treating the patient with an appropriate therapeutic regimen for aggressive prostate cancer if the diagnosis of the patient correlates to aggressive prostate cancer or treating the patient with an appropriate therapeutic regimen for non-aggressive prostate cancer if the diagnosis of the patient correlates to non-aggressive prostate cancer.

In other aspects, a method for identifying a male of European descent as having or likely to have aggressive prostate cancer is provided. In some aspects, the method comprises the steps of: a) obtaining a biological sample from the patient; b) detecting expression levels in the biological sample of a group of prostate cancer driver genes, said genes comprising ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3; and c) determining the expression threshold level for each prostate cancer driver gene in step b. In aspects, determining the expression threshold level for each gene comprises: 1) measuring the amount or expression of each prostate cancer drive gene in step b or biomarkers in a statistically significant number of samples from male patients of European descent with the different aggressive prostate cancer statuses; and 2) utilizing a defined aggressive phenotype and non-aggressive phenotype of prostate cancer in predicting prostate cancer disease aggressiveness using recursive partitioning; d) determining a number of positive indications for aggressive prostate cancer by comparing the detected expression levels of each gene determined in step b) to the expression threshold level for each determined in step c) for each gene. The method further comprises the steps of: e) determining a gene score threshold using recursive partitioning; and f) identifying the patient as having or likely to have aggressive prostate cancer if there are more positive indications then the gene score threshold determined in step e).

In certain aspects of a method for identifying a male of European descent as having or likely to have aggressive prostate cancer, the aggressive phenotype comprises a Gleason score of greater than or equal to 8 or 7 (4+3), T3 disease and BCR within 3 years. In aspects of method for identifying a male of European descent as having or likely to have aggressive prostate cancer the non-aggressive phenotype comprises a Gleason score of less than or equal to 6, T2 disease, and no BCR within 5 years.

In certain aspects of a method for identifying a male of European descent as having or likely to have aggressive prostate cancer, the recursive partitioning of step c) 2) comprises two daughter nodes. In some aspects, the daughter nodes of the recursive partitioning of step c) 2) require at least 30% of the sample to be in both daughter nodes. In certain aspects, the recursive partitioning step of step e) comprises two daughter nodes. In some aspects, the daughter nodes of the recursive partitioning of step e) require at least 30% of the sample to be in both daughter nodes.

In certain aspects of a method for identifying a male of European descent as having or likely to have aggressive prostate cancer, determining the number of positive indications comprises determining if AKT1, ALOX12, ALOX15, CGA, CXCR4, CYP19A1, FASN, IL6, IL8, NFKB1, PIK3C3, PIK3CA, TGFB1, and TIMP3 are upregulated in the biological sample to a level greater than the expression threshold level for each gene and determining if at least one or more of ADIPOQ, ALOX15B, BMP2, ERG, IL1B, PIK3R1, and PLA2G2A are down regulated in the biological sample to a level less than the expression threshold level for each gene. In certain aspects of a method for identifying a male of European descent as having or likely to have aggressive prostate cancer, the method further comprises identifying the patient as having or likely to have nonaggressive prostate cancer if there are there are fewer positive indications than the gene score threshold determined in step e).

In certain aspects of a method for identifying a male of European descent as having or likely to have aggressive prostate cancer, the gene threshold score is ten.

In certain aspects of a method for identifying a male of European descent as having or likely to have aggressive prostate cancer, wherein the biological sample is a tumor biopsy.

In certain aspects of a method for identifying a male of European descent as having or likely to have aggressive prostate cancer, the step of detecting comprises PCR, DASL, genome wide RNA sequencing, targeted RNA sequencing, or an immunoassay.

In certain aspects of a method for identifying a male of European descent as having or likely to have aggressive prostate cancer, the method further comprises treating the patient with an appropriate therapeutic regimen for aggressive prostate cancer if the diagnosis of the patient correlates to aggressive prostate cancer or treating the patient with an appropriate therapeutic regimen for non-aggressive prostate cancer if the diagnosis of the patient correlates to non-aggressive prostate cancer.

In further aspects, a method for identifying a male of African descent as having or likely to have aggressive prostate cancer is provided. In some aspects, the methods comprise the steps of: a) obtaining a biological sample from the patient; b) detecting expression levels in the biological sample of a group of prostate cancer driver genes, said genes comprising ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3; c) determining a number of positive indications for aggressive prostate cancer by comparing the detected expression levels of each of the prostate cancer driver genes determined in step b to an expression threshold level for each prostate cancer driver gene; and d) identifying the patient as having or likely to have aggressive prostate cancer if there are more positive indications than a gene score threshold.

In other aspects, a method for identifying a male patient of European descent as having or likely to have aggressive prostate cancer is provided. In some aspects, the method comprises the steps of: a) obtaining a biological sample from the patient; b) detecting expression levels in the biological sample of a group of prostate cancer driver genes, said genes comprising ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3; c) determining a number of positive indications for aggressive prostate cancer by comparing the detected expression levels of each of the prostate cancer driver genes determined in step b to an expression threshold level for each prostate cancer driver gene; and d) identifying the patient as having or likely to have aggressive prostate cancer if there are more positive indications than a gene score threshold.

Methods of aiding in diagnosis, assessment and treatment according to aspects of the present disclosure include initiating or modifying a course of treatment administered or recommended to the subject based on results of one or more assays of one or more pro-inflammatory factors, and/or, one or more biomarkers, of the present disclosure.

The power of a diagnostic test to correctly predict or determine status is commonly measured as the sensitivity of the assay, the specificity of the assay, or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative. Diagnostic tests that use the pro-inflammatory factors, genes, and/or biomarkers as identified herein may show an ROC of at least 0.6, at least about 0.7, at least about 0.8, or at least about 0.9.

According to aspects of the present disclosure, an anti-cancer treatment is administered or recommended to the subject based on results of one or more assays of one or more pro-inflammatory factors, and/or one or more biomarkers, of the present disclosure. According to aspects, a more or less rigorous anti-cancer treatment is administered or recommended to the subject based on results of one or more assays of one or more pro-inflammatory factors, and/or one or more biomarkers, of the present disclosure.

According to aspects, an anti-cancer treatment of the prostate gland is administered or recommended to the subject based on results of one or more assays of one or more pro-inflammatory factors, and/or one or more biomarkers, of the present disclosure. According to aspects, a more or less rigorous anti-cancer treatment of the prostate gland is administered or recommended to the subject based on results of one or more assays of one or more pro-inflammatory factors, and/or one or more biomarkers, of the present disclosure.

In particular aspects, where results of one or more assays of one or more pro-inflammatory factors, and/or one or more biomarkers, of the present disclosure determines the cancer to be aggressive, a more rigorous anti-cancer treatment is administered or recommended to the subject based on results of one or more assays of one or more pro-inflammatory factors, and/or one or more biomarkers, of the present disclosure.

A less rigorous anti-cancer treatment includes, but is not limited to, less frequent active surveillance of the subject to detect abnormalities of the prostate and/or worsening or progression of prostate cancer. Such active surveillance can include: one or more additional assays of one or more pro-inflammatory factors, and/or one or more gens and/or biomarkers of the present disclosure; assay of prostate specific antigen, assay of androgen receptor, digital rectal exam, and biopsy.

A more rigorous anti-cancer treatment includes, but is not limited to, more frequent or earlier active surveillance of the subject to detect abnormalities of the prostate and/or worsening or progression of prostate cancer. A more rigorous course of anti-cancer treatment includes administration of one or more inhibitors of one or more pro-inflammatory factors of the present disclosure.

According to aspects of the present disclosure, a score of 10 for EAM and of 11 for AAM is indicative of aggressive prostate cancer.

Inhibitors

The term “inhibitor” refers to a substance having activity to specifically inhibit a specified molecule, e.g. a pro-inflammatory factor, particularly a pro-inflammatory chemokine, pro-inflammatory cytokine, pro-inflammatory cytokine receptor, or a pro-inflammatory chemokine receptor. An inhibitor reduces a level and/or activity of its target by at least 10%, 20%, 30%, 40%, 50%, 60%, 70%, 80%, 90%, 95%, 99% or more, compared to a control, such as absence of the inhibitor.

An inhibitor may reduce expression of its target. The term “expression,” and grammatical equivalents, refers to transcription of a gene to produce a corresponding mRNA and/or translation of the mRNA to produce the corresponding protein.

An inhibitor can be any molecule, chemical entity, biological entity, composition, or agent, having activity to specifically inhibit a specified molecule. An inhibitor can be, without limitation, an antibody, an antigen-binding antibody fragment, an aptamer, an enzyme, a gene editing agent, an antibody mimetic, a small molecule, a nucleic acid, an organic or inorganic molecule, or a combination of any two or more thereof. Inhibitors can be obtained by known methods, such as chemical synthesis, production by recombinant molecular biology methods, purified from existing materials, or obtained commercially.

Nucleic Acid Inhibitors

According to aspects of the present disclosure, the inhibitor is a nucleic acid. The term “nucleic acid” refers to RNA or DNA molecules having more than one nucleotide in any form including single-stranded, double-stranded, oligonucleotide or polynucleotide. Nucleic acid inhibitors include an antisense molecule, an aptamer, an RNA interference nucleic acid, such as, but not limited to, siRNA, shRNA, or microRNA; or a catalytic nucleic acid, such as, but not limited to, a DNAzyme, a ribozyme or a CRISPR/Cas knockdown construct directed to the target.

Nucleic acid inhibitors can be produced by chemical synthesis and/or using molecular biology techniques known in the art. For example, chemical synthesis of nucleic acid inhibitors is described in Herdewijn, P. (Ed.), Oligonucleotide Synthesis: Methods and Applications, Methods in Molecular Biology, Humana Press, 2004. Molecular biology methods relating to nucleic acid inhibitors are described, for example, in Sambrook, J. and Russell, D. W., Molecular Cloning: A Laboratory Manual, Cold Spring Harbor Laboratory Press, 3rd Ed, 2001; and Engelke, D. R., RNA Interference (RNAi): Nuts and Bolts of RNAi Technology, DNA Press LLC, Eagleville, Pa., 2003. Naturally occurring or modified nucleotides may be used in constructing a nucleic acid inhibitor. Modified nucleotides may be used to increase the stability of a nucleic acid inhibitor, increase resistance to nucleases, or enhance stability of binding to a target, for instance. Non-limiting examples of such modified nucleotides include phosphorothioates, phosphorodithioates boronophosphates, alkyl phosphonates such as methyl phosphonates, and phosphoramidates such as 3′-amino phosphoramidates.

Generally, antisense nucleic acids useful for inhibiting expression of a target are in the range of about 12 to about 100 nucleotides in length, but may be shorter or longer depending on the application.

According to aspects of the present disclosure, an inhibitor is a double-stranded RNA molecule that inhibits expression of a target gene by RNA interference.

RNA interference is a target sequence-specific method of inhibiting a selected gene. RNA interference has been characterized in numerous organisms and is known to be mediated by a double-stranded RNA, also termed herein a double-stranded RNA compound. Briefly described, RNA interference involves a mechanism triggered by the presence of small interfering RNA, siRNA, resulting in degradation of a target complementary mRNA, siRNA is double-stranded. RNA which includes a nucleic acid sequence complementary to a target sequence in the gene to be silenced. The double-stranded RNA may be provided as a long double-stranded RNA compound, in which case it is subject to cleavage by the endogenous endonuclease Dicer in a cell. Cleavage by Dicer results in siRNA duplexes having about 21-23 complementary nucleotides in each of the sense strand and the antisense strand, and optionally 1-2 nucleotide 3′ overhangs on each of the two strands.

Alternatively, siRNA is provided as a duplex nucleic acid having a sense strand and an antisense strand, wherein the sense and antisense strands are substantially complementary and each of the sense and antisense strands have about 16-30 nucleotides. The complementary sense and antisense strands and optionally include 1-2 nucleotide 3′ overhangs on one or both of the two strands. In one embodiment, an siRNA is preferred which has sense and antisense strands, wherein each of the two strands has 21-23 nucleotides, wherein 2 nucleotides on the 3′ end of each strand are overhanging and the remaining 19-21 nucleotides are 100% complementary. As noted above, further details of siRNA compounds are described in Engelke, D. R., RNA Interference (RNAi): Nuts and Bolts of RNAi Technology, DNA Press LLC, Eagleville, Pa., 2003 Additional description of siRNA length and composition is found in Elbashir, S. M. et al., Genes and Devel., 15:188-200, 2001; and O'Toole, A. S. et al., RNA, 11:512-516, 2005.

siRNA provided as a duplex nucleic acid having a sense strand and an antisense strand may be configured such that the sense strand and antisense strand form a duplex in hybridization conditions but are otherwise unconnected. A double-stranded siRNA compound may be assembled from separate antisense and sense strands. Thus, for example, complementary sense and antisense strands are chemically synthesized and subsequently annealed by hybridization to produce a synthetic double-stranded siRNA compound.

Further, the sense and antisense strands for inclusion in siRNA may be produced from one or more expression cassettes encoding the sense and antisense strands. Where the sense and antisense strands are encoded by a single expression cassette, they may be excised from a produced transcript to produce separated sense and antisense strands and then hybridized to form a duplex siRNA. See, for example, Engelke, D. R, RNA Interference (RNAi): Nuts and Bolts of RNAi Technology, particularly chapters 5 and 6, DNA Press LLC, Eagleville, Pa., 2003 for further details of synthetic and recombinant methods of producing siRNA.

In a further alternative, a double-stranded “short hairpin” RNA compound, termed “shRNA” or “hairpin siRNA” includes an antisense strand and a sense strand connected by a linker, shRNA may be chemically synthesized or formed by transcription of a single-stranded RNA from an expression cassette in a recombinant nucleic acid construct. The shRNA has complementary regions which form a duplex under hybridization conditions, forming a “hairpin” conformation wherein the complementary sense and antisense strands are linked, such as by a nucleotide sequence of about 1-20 nucleotides. In general, each of the complementary sense and antisense strands have about 16-30 nucleotides.

As noted, siRNA and shRNA may be expressed from a double-stranded DNA template encoding the desired transcript or transcripts. A double-stranded DNA template encoding the desired transcript or transcripts is inserted in a vector, such as a plasmid or viral vector, and operably linked to a promoter for expression in vitro or in vivo. Plasmids and viral vectors suitable for transcription of a double-stranded DNA template are known in the art. Particular viral vectors illustratively include those derived from adenovirus, adeno-associated virus and lentivirus.

Optionally, an inhibitor is a CRISPR/Cas knockdown construct directed to the target, see for example Yin H. et al. Therapeutic genome editing by combined viral and non-viral delivery of CRISPR system components in vivo. Nat Biotechnol, 2016, 34(3):328-33; and Sachdeva M. et al. CRISPR/Cas9: molecular tool for gene therapy to target genome and epigenome in the treatment of lung cancer, Cancer Gene Ther., 2015, 22(11):509-17.

Antibody Inhibitors

The term “antibody′” is used herein in its broadest sense and includes, without limitation, single antibodies and mixtures of antibodies, and antigen binding fragments, characterized by substantially specific binding to an antigen. An antibody provided according to compositions and methods is illustratively a polyclonal antibody, a monoclonal antibody, a chimeric antibody, a humanized antibody, and/or an antigen-binding antibody fragment, for example. The term antibody refers to a standard intact immunoglobulin having four polypeptide chains including two heavy chains (H) and two light chains (L) linked by disulfide bonds in particular embodiments. Antigen binding antibody fragments illustratively include an Fab fragment, an Fab′ fragment, an F(ab′)2 fragment, an Fd fragment, an Fv fragment, an scFv fragment and a domain antibody (dAb), for example. In addition, the term antibody refers to antibodies of various classes including IgG, IgM, IgA, IgD and IgE, as well as subclasses, illustratively including for example human subclasses IgG1, IgG2, IgG3 and IgG4 and marine subclasses IgG1, IgG2, IgG2a. IgG2b, IgG3 and IgGM, for example.

Antibodies, antigen-binding fragments and methods for their generation are known in the art, for instance, as described in Antibody Engineering, Kontemann, R. and Dubel, S. (Eds.), Springer, 2001; Harlow, E. and Lane, D., Antibodies: A Laboratory Manual, Cold Spring Harbor Laboratory Press, 1988; Ausubel. F. et al., (Eds.), Short Protocols in Molecular Biology, Wiley, 2002; J. D. Pound (Ed.) Immunochemical Protocols, Methods in Molecular Biology, Humana Press, 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975).

An inhibitor according to aspects of the present disclosure may be an aptamer. The term “aptamer” refers to a nucleic acid that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Techniques for identification and generation of aptamers is known in the art as described, for example, in F. M, Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3rd Ed., 2001.

Particular TNF-α inhibitors include: infliximab, adalimumab, etanercept, golimumab, certrolizumab, TNF-related apoptosis-inducing ligand, thalidomide, pomalidomide, lenalidomide, a premilast, prednisone, efalizumab, ustekinumab, beclomethasone, betamethasone, cortisone, dexamethasone, hydrocortisone, methylprednisolone, prednisolone. In other aspects, TNF-α inhibitor may include one or more of infliximab, adalimumab, etanercept, golimumab, certrolizumab, TNF-related apoptosis-inducing ligand, thalidomide, pomalidomide, lenalidomide, apremilast, prednisone, efalizumab, and ustekinumab.

Particular IL-1β inhibitors include: anakinra, canakinumab, and rilonacept.

Particular IL-6 inhibitors include: tocilizumab, sarilumab, and satralizumab.

Particular IL-8 inhibitors include: BMS-986253 (abti-IL-8 mAb) and reparixin.

A particular IL-10 inhibitor is rituximab.

Particular ENA-78 inhibitors include LY294002

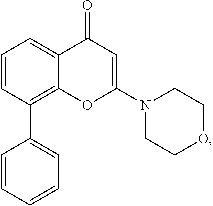 [SMILES: O=c1cc(N2CCOCC2)oc2c(-c3ccccc3)cccc12]

### and SB225002

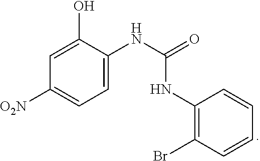 [SMILES: O=C(Nc1ccc([N+](=O)[O-])cc1O)Nc1ccccc1Br]

Particular RAGE inhibitors include azeliragon.

Particular MMP9 inhibitors include JNJ0966

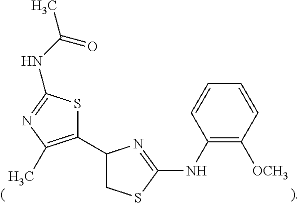 [SMILES: COc1ccccc1NC1=NC(c2sc(NC(C)=O)nc2C)CS1]

Particular HER2 inhibitors include: trastuzumab, lapatinib, neratinib, margetuximab, pertuzumab, tucatinib, and dacomitinib.

Particular CXCR4 inhibitors include: periflaxor, AMD3100

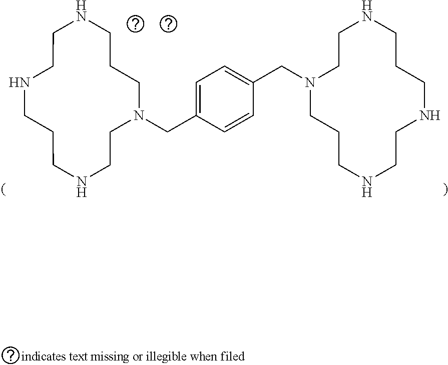 [SMILES: c1cc(CN2CCCNCCNCCCNCC2)ccc1CN1CCCNCCNCCCNCC1]

### and AMD11070

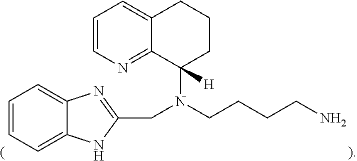 [SMILES: NCCCCN(Cc1nc2ccccc2[nH]1)[C@H]1CCCc2cccnc21]

Additional Therapeutic Modalities

Combinations of an inhibitor and one or more additional therapeutic agents are administered to a subject in need thereof according to aspects of the present invention.

The term “additional therapeutic agent” is used herein to refer to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included according to aspects of methods and compositions of the present invention include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal anti-inflammatory agents, steroids and vasoactive agents.

An additional pharmaceutical agent is an anti-cancer agent according to aspects of the present invention.

Anti-cancer agents are described, for example, in Goodman et al., Goodman and Gilman's The Pharmacological Basis of Therapeutics, 8th Ed., Macmillan Publishing Co., 1990.

Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bevacizumab, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, cobimetinib, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, vemurafenib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

An anti-cancer agent can be an immune checkpoint inhibitor.

Optionally, a method of treating cancer in a subject in need thereof further includes an adjunct anti-cancer treatment. An adjunct anti-cancer treatment can be a radiation treatment of a subject or an affected area of a subject's body. An adjunct anti-cancer treatment can be a chemical or surgical castration, for example.

An additional therapeutic treatment or adjunct anti-cancer treatment can include, without limitation, cryotherapy, external beam radiotherapy, hormonal therapy, interstitial prostate brachytherapy, radical prostatectomy and pharmaceutical therapies such as, but not limited to, administration of an anticholinergic agent; administration of an alpha-1-adrenergic receptor antagonist (also called alpha blockers), illustratively including alfuzosin, doxazosin, silodosin, tamsulosin, and terazosin; administration of a 5-alpha-reductase inhibitor, illustratively including dutasteride and finasteride; or a combination of two or more of an alpha-blocker, a 5-alpha-reductase inhibitor and an anticholinergic agent.

Administration

Methods of the present invention include administration of a pharmaceutical composition including an inhibitor of the present disclosure by a route of administration including, but not limited to, oral, rectal, nasal, pulmonary, epidural, ocular, otic, intraarterial, intracardiac, intracerebroventricular, intradermal, intravenous, intramuscular, intraperitoneal, intraosseous, intrapulmonary, intrathecal, intratumoral, intravesical, ophthalmic, parenteral, subcutaneous, topical, transdermal, and transmucosal, such as by sublingual, buccal, vaginal, and inhalational, routes of administration. Methods of the present invention include administration of a pharmaceutical composition including an inhibitor of the present disclosure by direct administration to the prostate.

The dosage of an inhibitor and any optional additional therapeutic agent will vary based on factors such as, but not limited to, the route of administration; the age, health, sex, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

According to aspects of the present invention, assays for effects of inhibitors of pre-inflammatory factors are used to monitor a subject. Thus, for example, a test sample is obtained from the subject before treatment according to a method of the present disclosure and at one or more times during and/or following treatment in order to assess effectiveness of the treatment. In a further example, a test sample is obtained from the subject at various times in order to assess the course or progress of disease or healing.

Methods of treating cancer are provided according to aspects of the present disclosure which include obtaining a first sample containing or suspected of containing one or more pro-inflammatory factors from the subject prior to administering one or more inhibitors of the one or more pro-inflammatory factors; obtaining a second sample containing or suspected of containing the one or more pro-inflammatory factors from the subject after administering the one or more inhibitors of the one or more pro-inflammatory factors; and assaying the first and second samples for the one or more pro-inflammatory factors, wherein a decrease in at least one of the pro-inflammatory factors is an indicator of an anti-cancer effect of treatment, thereby monitoring effectiveness of administering the one or more inhibitors.

Methods of treating prostate cancer are provided according to aspects of the present disclosure which include obtaining a first sample containing or suspected of containing one or more pro-inflammatory factors from the subject prior to administering one or more inhibitors of the one or more pro-inflammatory factors; obtaining a second sample containing or suspected of containing the one or more pro-inflammatory factors from the subject after administering the one or more inhibitors of the one or more pro-inflammatory factors; and assaying the first and second samples for the one or more pro-inflammatory factors, wherein a decrease in at least one of the pro-inflammatory factors is an indicator of an anti-prostate cancer effect of treatment, thereby monitoring effectiveness of administering the one or more inhibitors.

### Pharmaceutical Compositions

In some aspects, the inhibitors as described herein can be administered to a subject as part of a pharmaceutical composition. A composition or a pharmaceutical composition according to aspects of the present invention includes about 0.1-99% of an inhibitor, and optionally a pharmaceutically acceptable carrier.

A pharmaceutical composition of the present disclosure may be in any dosage form suitable for administration to a subject, illustratively including solid, semi-solid and liquid dosage forms such as tablets, capsules, powders, granules, suppositories, pills, solutions, suspensions, ointments, lotions, creams, gels, pastes, sprays and aerosols. Liposomes and emulsions are well-known types of pharmaceutical formulations that can be used to deliver a pharmaceutical agent, particularly a hydrophobic pharmaceutical agent. Pharmaceutical compositions of the present invention generally include a pharmaceutically acceptable carrier such as an excipient, diluent and/or vehicle. Delayed release formulations of compositions and delayed release systems, such as semipermeable matrices of solid hydrophobic polymers can be used.

The term “pharmaceutically acceptable carrier” may refer to a carrier which is suitable for use in a subject without undue toxicity or irritation to the subject and which is compatible with other ingredients included in a pharmaceutical composition. Pharmaceutically acceptable carriers, methods for making pharmaceutical compositions and various dosage forms, as well as modes of administration are well-known in the art, for example as detailed in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A solid dosage form for administration or for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alginates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms may optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present invention to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include one or more active agents and a pharmaceutically acceptable carrier formulated as an emulsion, solution, suspension, syrup, or elixir. A liquid dosage form of a composition of the present invention may include a colorant, a stabilizer, a wetting agent, an emulsifying agent, a suspending agent, a sweetener, a flavoring, or a perfuming agent.

For example, a composition for parenteral administration may be formulated as an injectable liquid. Examples of suitable aqueous and nonaqueous carriers include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

For topical administration, a composition can be formulated for administration to the skin such as for local effect, and/or as a “patch” formulation for transdermal delivery. Pharmaceutical formulations suitable for topical administration include, for example, ointments, lotions, creams, gels, pastes, sprays and powders. Ointments, lotions, creams, gels and pastes can include, in addition to one or more active agents, a base such as an absorption base, water-removable base, water-soluble base or oleaginous base and excipients such as a thickening agent, a gelling agent, a colorant, a stabilizer, an emulsifying agent, a suspending agent, a sweetener, a flavoring, or a perfuming agent.

Transdermal formulations can include percutaneous absorption enhancers such as acetone, azone, dimethyl acetamide, dimethyl formamide, dimethyl sulfoxide, ethanol, oleic acid, polyethylene glycol, propylene glycol and sodium lauryl sulfate. Ionotophoresis and/or sonophoresis can be used to enhance transdermal delivery.

Powders and sprays for topical administration of one or more active agents can include excipients such as talc, lactose and one or more silicic acids. Sprays can include a pharmaceutical propellant such as a fluorinated hydrocarbon propellant, carbon dioxide, or a suitable gas. Alternatively, a spray can be delivered from a pump-style spray device which does not require a propellant. A spray device delivers a metered dose of a composition contained therein, for example, using a valve for regulation of a delivered amount.

Ophthalmic formulations of one or more active agents can include ingredients such as a preservative, a buffer and a thickening agent.

Suitable surface-active agents useful as a pharmaceutically acceptable carrier or excipient in the pharmaceutical compositions of the present invention include non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphthalene sulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphosphatidyl-choline and their mixtures.

Suitable non-ionic surfactants useful as pharmaceutically acceptable carriers or excipients in the pharmaceutical compositions of the present invention include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants useful as pharmaceutically acceptable carriers or excipients in the pharmaceutical compositions of the present invention include quaternary ammonium salts, preferably halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2nd ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

In particular aspects, a pharmaceutically acceptable carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486.

A particulate pharmaceutically acceptable carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A pharmaceutically acceptable salt of an inhibitor can be used. The term “pharmaceutically acceptable salt” refers to salts which are suitable for use in a subject without undue toxicity or irritation to the subject and which are effective for their intended use.

Pharmaceutically acceptable salts include pharmaceutically acceptable acid addition salts and base addition salts. Pharmaceutically acceptable salts are well-known in the art, such as those detailed in S. M. Berge et al., J. Pharm. Sci., 66:1-19, 1977. Exemplary pharmaceutically acceptable salts are those suitable for use in a subject without undue toxicity or irritation to the subject and which are effective for their intended use which are formed with inorganic acids such as hydrochloric acid, hydrobromic acid, hydroiodic acid, nitric acid, phosphoric acid, sulfuric acid and sulfamic acid; organic acids such as acetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, formic acid, fumaric acid, glutamic acid, glycolic acid, glycerophosphoric acid, hemisulfic acid, heptanoic acid, hexanoic acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, trichloroacetic acid, trifluoroacetic acid and undecanoic acid; inorganic bases such as ammonia, hydroxide, carbonate, and bicarbonate of ammonium; organic bases such as primary, secondary, tertiary and quaternary amine compounds ammonium, arginine, betaine, choline, caffeine, diolamine, diethylamine, diethanolamine, 2-dimethylaminoethanol, 2-diethylaminoethanol, dicyclohexylamine, dicyclohexylamine, dibenzylamine, N, N-dibenzylphenethylamine, 1-ephenamine, N, N′-dibenzylethylenediamine, ethanolamine, ethylamine, ethylenediamine, glucosamine, histidine, hydrabamine, isopropylamine, 1H-imidazole, lysine, methylamine, N-ethylpiperidine, N-methylpiperidine, N-methylmorpholine, N, N-dimethylaniline, piperazine, trolamine, methylglucamine, purines, piperidine, pyridine, theobromine, tetramethylammonium compounds, tetraethylammonium compounds, trimethylamine, triethylamine, tripropylamine and tributylamine and metal cations such as aluminum, calcium, copper, iron, lithium, magnesium, manganese, potassium, sodium, and zinc.

Commercial Packages

Commercial packages are provided according to aspects of the present invention which include an inhibitor of a pro-inflammatory cytokine, chemokine, cytokine receptor, and/or chemokine receptor.

Instructions for administering the inhibitor of a pro-inflammatory cytokine, chemokine, cytokine receptor, and/or chemokine receptor are included according to aspects of the invention.

Commercial packages are provided according to aspects of the present invention which include one or more reagents for assay of any one, two, three, four, five, six, seven, eight, nine, ten, eleven, or all of: CXCR4, IL-8, IL-6, HER2, TNFα, IL-1β, SDF-1a, MMP-9, ENA-78, leptin, RAGE, and IL-10.

Commercial packages are provided according to aspects of the present invention which include one or more reagent for assay of any one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or all of: ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, TIMP3, MMP-9, STAT3 and TNF-α.

One or more ancillary components is optionally included in commercial packages of the present invention, such as a buffer or diluent.

Embodiments of inventive compositions and methods are illustrated in the following examples. These examples are provided for illustrative purposes and are not considered limitations on the scope of inventive compositions and methods.

### EXAMPLES

### Example 1

### Biological Sample Analysis

A panel of pro-inflammatory factors, which can be key contributors to racial disparities in PCa aggressiveness and outcomes is analyzed. A panel of pro-inflammatory factors analyxzed includes IL1β, TNF-α, IL-6, IL-8, SDF-1a (CXCL12a), MMP-9, ENA-78, RAGE, Leptin, and IL-1β, see Tables 1 and 2.

Table 1 shows results of assays of cytokines in plasma of newly diagnosed prostate cancer patients which were most differentially distributed based on race, EAM=European-American Men, AAM=African-American Men. Analyses were based on fluorescence intensity scores for each cytokine.

TABLE 1

| | Mean | | Median | | |
|---|---|---|---|---|---|
| Cytokine | EAM | AAM | EAM | AAM | P-value |
| ENA-78 | 270.2 | 3,355.3 | 119.5 | 267.0 | <0.001 |
| (CXCL5) | | | | | |
| IL-10 | 30.7 | 401.1 | 4.4 | 5.1 | <0.001 |
| IL-6 | 67.4 | 111.3 | 40.5 | 37.8 | 0.002 |
| Leptin | 22,087.2 | 48,759.1 | 3,262.4 | 3,382.7 | 0.008 |
| RAGE | 701.4 | 864.6 | 639.8 | 462.6 | 0.022 |

Table 2 shows results of assays of cytokines in plasma of newly diagnosed prostate cancer patients which were most differentially distributed based on Disease Aggressiveness. Analyses were based on fluorescence intensity scores for each cytokine.

TABLE 2

| | Mean | | Median | | |
|---|---|---|---|---|---|
| | Non- | | Non- | | |
| Cytokine | aggressive | Aggressive | aggressive | Aggressive | P-value |
| RAGE | 621.6 | 1,214 | 452.7 | 670.1 | <0.001 |
| IL-6 | 82.38 | 134.6 | 34.7 | 44.3 | 0.001 |
| ENA-78 | 1,372.9 | 3,184.6 | 200.8 | 249.5 | 0.028 |
| (CXCL5) | | | | | |
| TNFa | 199.9 | 265.8 | 152.3 | 156.7 | 0.026 |
| IL-8 | 88.8 | 111.5 | 71.7 | 70.7 | 0.046 |

Based on the presently disclosed examination of plasma samples from prostate cancer (PCa) patients, IL-10 levels are much higher in AAM patients as well as PCa patients with aggressive PCa, as shown in Tables 1 and 2. Leptin levels are significantly higher in AAM as shown in Table 1.

Accordingly, the combined presence of several of pro-inflammatory factors, rather than a single factor, can predict aggressiveness of the disease in a patient diagnosed with PCa. Thus, a “proinflammatory marker score” based on the cumulative number and plasma levels of these pro-inflammatory factors as measured by an assay, was determined, exemplified in this example by a quantitative fluorescent antibody array. This proinflammatory marker score can be then used to define PCa aggressiveness in each individual in a similar manner as previously demonstrated with tissue analysis in radical prostatectomies where defined gene signatures and gene scores were utilized to establish a cut-point for aggressive versus non-aggressive disease.

A preliminary examination of 15 cytokines (IL-8, IL-10, IL-18, RAGE, RANTES, MCP-1, Visfatin, Resistin, ENA-78 and VCAM-1, CRP, TNF-α, leptin, IL-6 and HGF) in a sub-set of 69 newly diagnosed PCa patients (53 AAM, 16 EAM) using a Custom Fluorescent Quantitative Antibody Arrays, a glass slide array-based multiplex sandwich ELISA system (commercially available from RayBiotech, Norcross, Ga.) was performed. A quantifiable amount of each target pro-inflammatory factor is determined by the intensity of a fluorescent signal based on an 8-point standard curve for each target pro-inflammatory factor. The use of Quantibody array-specific Q Analyzer® software allows data quantification and analysis. In the initial analyses of a subset of 69 patient plasma samples, the fluorescence intensity values for each of the 15 cytokines were determined and it was identified that there are significant differences in the levels of 7 circulating pro-inflammatory factors depending on race and/or aggressiveness of the disease as demonstrated in Tables 1 and 2.

Plasma samples can be processed and analyzed according to manufacturer's instructions (RayBiotech, Norcross, Ga.). This system was used for preliminary examination of 69 plasma samples and results are shown in Tables 1 and 2). The analysis of each plasma sample using this custom 10-cytokine array generates 4 fluorescence intensity values which correspond to a concentration of the assayed proinflammatory factor per ml of plasma. Each sample can be analyzed on two separate arrays to account for potential inter-assay variability. Q Analyzer® software can then convert the fluorescence intensity value to a circulating concentration of each cytokine. The values obtained are then used to establish the “proinflammatory marker score”.

### Example 2

To derive a gene score, the investigators examined twenty-one functionally related, prostate cancer driver genes that had a statistically significant difference in expression in males of African descent and males of European descent: ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3. Utilizing the aggressive and nonaggressive phenotypes as follows: aggressive prostate cancer includes GS=8 or 7 (4+3), T3 disease and BCR within 3 years; non-aggressive prostate cancer includes GS=6, T2 disease, and no BCR within 5 years, the expression threshold for each gene individually by race was identified in predicting disease aggressiveness using recursive partitioning, requiring at least 30% of the sample to be in both of the daughter nodes. This resulted in a high-risk and a low-risk subset for each gene for each race. The online database for the data generated from the DASL assay used for determining the expression thresholds for each gene by race is found at http://www.ncbi.nlm.nih.gov/geo/query/acc.cgi?acc=GSE41968.

For males of African descent, if the genes or biomarkers comprising ADIPOQ, AKT1, ALOX12, ALOX15, BMP2, CGA, CXCR4, CYP19A1, FASN, IL1B, IL8, NFKB1, PLA2G2A TGFB1, and TIMP3 are up-regulated compared to normalized expression values (e.g. normalized gene expression values in no cancer or non-aggressive prostate cancer patients), then a measured amount(s) above the expression threshold for each gene or biomarker provides a positive indication of aggressive prostate cancer. Additionally, if the genes or biomarkers comprising ALOX15B, ERG, IL6, PIK3C3, PIK3CA, and PIK3R1, are down-regulated in males of African descent compared to normalized expression values (e.g., normalized gene expression values in no cancer or non-aggressive prostate cancer patients), then a measured amount for each gene or biomarker below the expression threshold provides a positive indication of aggressive prostate cancer. The expression thresholds for each of these twenty-one genes in males of African descent using the DASL assay are disclosed in Table 3. Recursive partitioning is used to determine the expression threshold for ADIPOQ in males of African descent.

For males of European descent, if the genes or biomarkers comprising AKT1, ALOX12, ALOX15, CGA, CXCR4, CYP19A1, FASN, IL6, IL8, NFKB1, PIK3C3, PIK3CA, TGFB1, and TIMP3 are up-regulated compared to normalized expression values (e.g. normalized gene expression values in no cancer or non-aggressive prostate cancer patients), then a measured amount above the expression threshold for each gene or biomarker provides a positive indication of aggressive prostate cancer. Additionally, if the genes or biomarkers comprising ADIPOQ, ALOX15B, BMP2, ERG, IL1B, PIK3R1, and PLA2G2A are down-regulated in males of European descent compared to normalized expression values (e.g. normalized gene expression values in no cancer or non-aggressive prostate cancer patients), then a measured amount below the expression threshold for each gene or biomarker provides a positive indication of aggressive prostate cancer. The expression thresholds for each of these twenty-one genes in males of European descent using the DASL assay are disclosed in Table 3.

Subsequently, the number of genes that a patient has that are high risk (e.g., provides a positive indication of cancer based on the expression threshold determined for the specific assay used) are summed to create a gene score. Recursive partitioning is again used to define a threshold for the gene score for each race. These gene scores for males of African descent and males of European descent are based on the results of DASL microarray gene expression analysis. In this example race-specific thresholds for prostate cancer aggressiveness and treatment decision can be determined: if an a male patient of African descent had at least 11 of 21 genes that were high-risk they were determined to have aggressive prostate cancer, while if a male patient of European descent had at least 12 of 21 genes that were high-risk they were determined to have aggressive prostate cancer. Using the DASL assay, the resultant sensitivity and specificity for males of African descent with 11 or more high risk genes is 100% and 69%, respectively. The resultant sensitivity and specificity for males of European descent with 10 or more high risk genes is 88% and 85%, respectively.

Definition of high risk subset for each gene and race. For example for ADIPOQ, normalized gene expression values>−3.343 for males of African descent are predicted to be aggressive; while for males of European descent normalized gene expression values<−3.322 are predicted to be aggressive.

TABLE 3
Determined expression thresholds for twenty-one functionally
related, prostate cancer driver genes in males of African descent
and males of European descent using the DASL assay.

| Gene | AAM.dir | AAM.cutpt | EAM.dir | EAM.cutpt |
|---|---|---|---|---|
| ADIPOQ | > | −3.343 | < | −3.322 |
| AKT1 | > | −1.687 | > | −1.707 |
| ALOX12 | > | −4.034 | > | −4.038 |
| ALOX15 | > | −3.463 | > | −3.479 |
| ALOX15B | < | −0.400 | < | −0.679 |
| BMP2 | > | −2.327 | < | −2.985 |
| CGA | > | −4.063 | > | −4.658 |
| CXCR4 | > | −0.422 | > | −0.505 |
| CYP19A1 | > | −4.027 | > | −4.523 |
| ERG | < | −2.270 | < | −2.005 |
| FASN | > | −0.496 | > | −0.197 |
| IL1B | > | −1.354 | < | −1.974 |
| IL6 | < | −0.722 | > | −1.516 |
| IL8 | > | −0.596 | > | −1.201 |
| NFKB1 | > | −0.228 | > | −0.174 |
| PIK3C3 | < | −0.053 | > | −0.058 |
| PIK3CA | < | −0.247 | > | −0.197 |
| PIK3R1 | < | 0.238 | < | 0.625 |
| PLA2G2A | > | 1.211 | < | 1.199 |
| TGFB1 | > | −0.196 | > | −0.285 |
| TIMP3 | > | −3.551 | > | −3.407 |

Any patents or publications mentioned in this specification are incorporated herein by reference to the same extent as if each individual publication is specifically and individually indicated to be incorporated by reference. U.S. patent application Ser. No. 16/071,325 is incorporated herein by reference in its entirely.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of treatment of a subject having, or suspected of having, cancer, comprising:
inhibiting two or more of: CXCR4, IL-8, IL-6, HER2, TNFα, IL-1β, SDF-1a, MMP-9, ENA-78, leptin, RAGE, and IL-10 in the subject,
with the proviso that, when CXCR4 and TNFα are inhibited, at least one additional member of the group: IL-8, IL-6, HER2, IL-1β, SDF-1a, MMP-9, ENA-78, leptin, RAGE, and IL-10 is also inhibited;
with the proviso that, when CXCR4 and SDF-1a are inhibited, at least one additional member of the group: IL-8, IL-6, HER2, IL-1β, TNFα, MMP-9, ENA-78, leptin, RAGE, and IL-10 is also inhibited;
with the proviso that, when IL-8, IL-6, and TNFα are inhibited, at least one additional member of the group: CXCR4, HER2, SDF-1a, MMP-9, ENA-78, leptin, RAGE, and IL-10 is also inhibited; and
with the proviso that when IL-8, IL-6, TNFα, and IL-1β are inhibited, at least one additional member of the group: CXCR4, HER2, SDF-1a, MMP-9, ENA-78, leptin, RAGE, and IL-10 is also inhibited.

2. The method of treatment of claim 1, comprising:
inhibiting two or more of: IL-8, IL-6, HER2, TNFα, IL-1β, SDF-1a, MMP-9, ENA-78, leptin, RAGE, and IL-10 in the subject,
with the proviso that, when IL-8, IL-6, and TNFα are inhibited, at least one additional member of the group: HER2, SDF-1a, MMP-9, ENA-78, leptin, RAGE, and IL-10 is also inhibited; and
with the proviso that when IL-8, IL-6, TNFα, and IL-1β are inhibited, at least one additional member of the group: HER2, SDF-1a, MMP-9, ENA-78, leptin, RAGE, and IL-10 is also inhibited.

3. The method of treatment of claim 1, comprising:
inhibiting two or more of: IL-8, IL-6, TNFα, ENA-78, leptin, RAGE, and IL-10 in the subject,
with the proviso that, when IL-8, IL-6, and TNFα are inhibited, at least one additional member of the group: ENA-78, leptin, RAGE, and IL-10 is also inhibited.

4. The method of treatment of claim 1, wherein the inhibiting comprises administration of an effective amount of at least one inhibitor.

5. The method of treatment of claim 3, wherein the inhibitor comprises a nucleic acid inhibitor, an antibody, an antigen-binding antibody fragment, an aptamer, an inorganic or organic small molecule inhibitor, or a combination of any two or more thereof.

6. The method of treatment of claim 1, wherein the cancer is prostate cancer.

7. The method of treatment of claim 6, wherein the prostate cancer is castration resistant prostate cancer.

8. The method of treatment of claim 3, wherein the administration is a systemic administration.

9. The method of treatment of claim 3, wherein the administration is a local administration.

10. The method of treatment of claim 1, further comprising assaying one or more of: CXCR4, IL-8, IL-6, HER2, TNFα, IL-1β, SDF-1a, MMP-9, ENA-78, leptin, RAGE, and IL-10 in a subject sample.

11. The method of treatment of claim 10, wherein the assaying comprises assaying one or more of CXCR4, IL-8, IL-6, HER2, TNFα, IL-1β, SDF-1a, MMP-9, ENA-78, leptin, RAGE, and IL-10 in a subject sample obtained from the subject prior to treatment inhibiting two or more of: CXCR4, IL-8, IL-6, HER2, TNFα, IL-1β, SDF-1a, MMP-9, ENA-78, leptin, RAGE, and IL-10 in the subject.

12. The method of treatment of claim 10, wherein assaying one or more of CXCR4, IL-8, IL-6, HER2, TNFα, IL-1β, SDF-1a, MMP-9, ENA-78, leptin, RAGE, and IL-10 in a subject sample produces an assay result determining that one or more of: CXCR4, IL-8, IL-6, HER2, TNFα, IL-1β, SDF-1a, MMP-9, ENA-78, leptin, RAGE, and IL-10 is dysregulated in the subject.

13. The method of treatment of claim 10, wherein the assaying comprises assaying one or more of CXCR4, IL-8, IL-6, HER2, TNFα, IL-1β, SDF-1a, MMP-9, ENA-78, leptin, RAGE, and IL-10 in a subject sample obtained from the subject following treatment inhibiting two or more of: CXCR4, IL-8, IL-6, HER2, TNFα, IL-1β, SDF-1a, MMP-9, ENA-78, leptin, RAGE, and IL-10 in the subject.

14. The method of treatment of claim 1, further comprising assaying at least one of: ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3 to determine an aggressive or non-aggressive phenotype of the cancer.

15. The method of treatment of claim 10, further comprising assaying at least one of: ADIPOQ, AKT1, ALOX12, ALOX15, ALOX15B, BMP2, CGA, CXCR4, CYP19A1, ERG, FASN, IL1B, IL6, IL8, NFKB1, PIK3C3, PIK3CA, PIK3R1, PLA2G2A, TGFB1, and TIMP3 to determine an aggressive or non-aggressive phenotype of the cancer.

16. The method of treatment of claim 1, further comprising assaying at least one of: ADIPOQ, AKT1, ALOX12, ALOX15, BMP2, CGA, CXCR4, CYP19A1, ERG, FASN, IL1B, IL6, IL8, NFKB1, PIK3C3, PIK3CA, PIK3R1, PLA2G2A, TGFB1, MMP-9, STAT3 and TNF-α to determine an aggressive or non-aggressive phenotype of the cancer.

17. The method of treatment of claim 10, further comprising assaying at least one of: ADIPOQ, AKT1, ALOX12, ALOX15, BMP2, CGA, CXCR4, CYP19A1, ERG, FASN, IL1B, IL6, IL8, NFKB1, PIK3C3, PIK3CA, PIK3R1, PLA2G2A, TGFB1, MMP-9, STAT3 and TNF-α to determine an aggressive or non-aggressive phenotype of the cancer.

18. A method of assessing aggressiveness of a cancer in a subject, comprising assaying one or more pro-inflammatory factors selected from the group consisting of: CXCR4, IL-8, IL-6, HER2, TNFα, IL-1β, SDF-1a, MMP-9, ENA-78, leptin, RAGE, and IL-10 in a subject sample; and
comparing a result of the assaying to a control or standard, wherein when at least one, at least two, at least three, at least four, at least five, at least six, at least seven, at least eight, at least nine, at least ten, at least eleven, or all of the pro-inflammatory factors are determined to be dysregulated, the cancer is determined to be more likely to be aggressive.
